(19) United States
(12) Patent Application Publication
(10) Pub. No.: US 2003/0232362 A1
(43) Pub. Date: Dec. 18, 2003

(54) Drug resistance-associated gene and use thereof

(75) Inventors: Hideya Komatani, Ibaraki (JP); Yoshikazu Hara, Ibaraki (JP); Hidehito Kotani, Ibaraki (JP); Rinako Nakagawa, Ibaraki (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: BANYU PHARMACEUTICAL CO., Tokyo (JP)

(21) Appl. No.: 10/405,806

(22) Filed: Apr. 3, 2003

Related U.S. Application Data
(63) Continuation of application No. PCT/JP01/08112, filed on Sep. 18, 2001.

(30) Foreign Application Priority Data
Oct. 3, 2000 (JP) 2000-303441

(51) Int. Cl. C12Q 1/68; G01N 33/574; C07H 21/04; C12P 21/02; C12N 5/06; C07K 14/47; A61K 31/7056; A61K 31/407

(52) U.S. Cl. 435/6; 435/7.23; 435/69.1; 435/320.1; 435/325; 514/410; 536/23.2; 514/43; 530/350

(57) ABSTRACT

The present invention relates to a protein which excretes cancer chemotherapeutic drugs from a cell and which confers drug-resistance on the cell, a DNA encoding the protein, a method for detecting a cell type resistant to cancer chemotherapeutic drugs, a method for screening an inhibitor to overcome the resistance to cancer chemotherapeutic drugs as well as utilizing this knowledge to improve the therapeutic treatment of cancer patients.

### CROSS-REFERENCE TO A RELATED APPLICATION

The present application is a continuation application of PCT/JP01/08112 filed on Sep. 18, 2001, the entire contents of which are incorporated herein by reference.

### BACKGROUND OF THE INVENTION

1. Field of the Invention

The present invention is useful in a field of pharmaceutical products. More specifically, the present invention relates to a novel protein which excretes cancer chemotherapeutic drugs from a cell and which confers drug-resistance on the cell, a DNA encoding the protein, a method for detecting a cell type resistant to cancer chemotherapeutic drugs, indication for selecting a cancer chemotherapeutic drug for a patient, and a method for screening an inhibitor to overcome the resistance to cancer chemotherapeutic drugs.

2. Discussion of the Background

The development of resistance to cancer chemotherapeutic drugs is a major problem in the treatment of cancer by conventional cytotoxic drugs. Tumors are sensitive to chemotherapeutic drugs at first, however, exhibit multidrug resistance afterward, and result in relapse. As a major cause for such chemotherapeutic drug resistance, there is a decrease of intracellular drug concentration by excretion of the drug from the cell. This mechanism depends on the expression of a transporter that excretes chemotherapeutic drugs out of the cancer cell. P-glycoprotein encoded on MDR1 gene (hereinafter referred to as “P-gp”) and a multidrug resistance-associated protein encoded on MRP gene (hereinafter referred to as “MRP”) have been reported as typical members of such transporters. P-gp is a molecular pump which has been previously known to be involved in multidrug resistance to multiple types of tumor, and MRP is a transporter that was shown to be involved in multidrug resistance to lung cancer at first and then revealed to be expressed in other types of cancer as well (Cole, S. P. C. et al., Science 258, 1650-1654 (1992); Slovak, M. L. et al., Cancer Res. 53, 3221-3225 (1993)).

Each of these proteins is a member of ABC (ATP-binding cassette) transporter superfamily, and a molecule that is localized in a cell membrane and transports its substrates utilizing an energy source such as ATP hydrolysis.

Recently, various new ABC family molecules have been discovered in succession and these molecular pumps that are suggested to be involved in drug resistance, in addition to P-gp and MRP, have been studied. Some of the molecules make up a sub-family referred to as ABCG2 (BCRP/MXR/ABCP). The genes for this sub-family were reported as ABCP that was specifically expressed in placenta (Allikmets, R. et al., Cancer Res. 58, 5337-5339 (1998)), BCRP that was isolated from a resistant cell line selected with adriamycin (Doyle, A. et al., Proc. Natl. Acad. Sci. U.S.A. 95, 15665-15670 (1998)), and MXR that was isolated from a resistant cell line selected with mitoxantrone (Mivake, K. et al., Cancer Res. 59, 8-13 (1999)). In these three kinds of genes, variations of 1 to 4 amino acid sequences derived from the nucleotide substitutions between the respective genes were observed.

From the analysis of the cell line which is produced by introducing and expressing the nucleotide sequence reported as BCRP into MCF-7 cell, expression of this gene was shown to confer resistance to mitoxantrone and adriamycin. Thus, the gene has been notable for a novel factor of multidrug resistance (Doyle, A. et al., Proc. Natl. Acad. Sci. U.S.A. 95, 15665-15670 (1998) and WO 99/40110).

In the above reports, however, there is only disclosed the relationship between cancer chemotherapeutic drugs having an anthraquinone skeleton such as adriamycin, daunorubicin and mitoxantrone and the above-mentioned genes. In addition, there is no disclosure or suggestion concerning the relationship between amino acid replacement of the genes and the substrate specificity.

Recently, it was reported that the expression of ABCG2 (BCRP/MXR/ABCP) sub-family might be correlated with the resistance to topotecan that does not have an anthraquinone skeleton (Maliepaard, M. et al., Cancer Res. 59, 4559-4563 (1999)), however, it was not proved distinctly.

On the other hand, with respect to the drug resistant mechanisms to the compounds represented by formula (I) (hereinafter referred to as “indolocarbazole compound”):

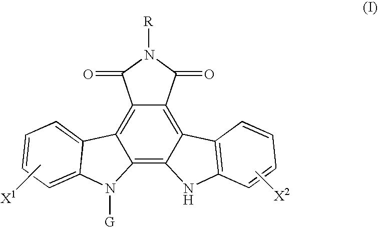

wherein X¹ and X² each independently represent a hydrogen atom, a halogen atom or a hydroxyl group; R represents a hydrogen atom, amino, formylamino, or lower alkylamino which may be substituted with any one selected from the group consisting of one to three hydroxyl group(s), a pyridyl group optionally having substituent(s), and thienyl group optionally having substituent(s); and G represents a pentose group, hexose group or derivative thereof, which may be substituted with an amino group. It has been reported that the growth inhibitory effect of Compound-A (in formula (I), X¹ represents 1-hydroxyl group, X² represents 11-hydroxyl group, R represents a formylamino and G represents β-D-glucopyranosyl group) to various cells was correlated with the accumulation of the Compound-A in the cells (Yoshinari, T. et al., Cancer Res. 55,1310-1315 (1995) and Kanzawa, F. et al., Cancer Res. 55, 2806-2813 (1995)).

In the above report, however, it was only disclosed that the resistance caused by the change of intracellular accumulation of the drug was not correlated with the function of the P-gp. Including all above reports, there is no report which has revealed a factor concerning the intracellular accumulation or drug resistance to the indolocarbazole compound in the past.

With respect to cancer chemotherapeutic drugs having an anthraquinone skeleton (adriamycin, doxorubicin, mitoxantrone and the like), the P-gp, MRP and BCRP are thought to be involved in resistance to the drugs. Therefore, these are not desirable cancer chemotherapeutic drugs. In view of such situations, it is desired to develop anti-cancer drugs that are not transported by P-gp and the like.

The above-mentioned compounds having an indolocarbazole skeleton were not reported to be involved in resistance by the P-gp, MRP and BCRP, so Compound-A (Yoshinari, T. et al., Cancer Res. 55,1310-1315 (1995)), and Compound-B (in the above general formula (I), X¹ represents 2-hydroxyl group, X² represents 10-hydroxyl group, R represents (1-hydroxymethyl-2-hydroxyl) ethylamino group, and G represents β-D-glucopyranosyl group) (Yoshinari, T. et al., Cancer Res. 59, 4271-4275 (1999)) both of which are topoisomerase I inhibitors, and UCN-01 which is a protein kinase inhibitor (Akinaga, S. et al., Cancer Res. 51, 4888-4892 (1991)) promise to be novel anticancer candidates. However, it is important to clarify a factor involved in the change of intracellular accumulation of indolocarbazole compounds in view of the above disclosure of Yoshinari et al.

### SUMMARY OF THE INVENTION

Accordingly, it is an object of the present invention to provide a protein which is relevant to the intracellular accumulation of indolocarbazole compounds and a polynucleotide coding therefor. It is also an object of the present invention to provide a method for detecting an expression of the gene in a cancer cell or cancer patient using the polynucleotide coding for the protein or an antibody against the protein. It is further an object of the present invention to provide a method for screening an inhibitor to overcome the resistance to cancer chemotherapeutic drugs using the gene and the protein.

### BRIEF DESCRIPTION OF THE DRAWINGS

FIG. 1 shows the expression of transcripts of the ABCG2 gene of the present application in Compound-A resistant cell lines.

FIG. 2 shows the expression of ABCG2 transcripts in PC-13 cell lines to which the ABCG2 gene of the present application was introduced.

FIG. 3 shows the accumulation of Compound-B in PC-13 cell lines to which the ABCG2 gene of the present application was introduced.

FIG. 4 shows the method of introducing an amino acid substitution in the ABCG2 gene using a two-step PCR: (a) a primer design at a base substitution site, (b) outline of the two-step PCR.

FIG. 5 shows the expression of each of ABCG2 transcripts in MCF-7 cells to which the ABCG2 gene of the present application or ABCG2-482T gene was introduced. In the drawing, “vector” represents MCF-7 cell with introduced the vector alone, “T8” represents MCF-7 cell with introduced ABCG2-482T(MCF-7/T8), and “R7” represents MCF-7 cell with introduced ABCG2 of the present application (MCF-7/R7).

FIG. 6 shows the relative resistance to each of anti-cancer drugs of MCF-7 cell to which ABCG2 of the present application or ABCG2-482T gene was introduced. In the drawing, “T8” represents MCF-7 cell with introduced ABCG2-482T (MCF-7/T8), and “R7” represents MCF-7 cell with introduced ABCG2 of the present application (MCF-7/R7).

FIG. 7 shows the amount of the accumulation of each of the drugs in MCF-7 cell to which ABCG2 of the present application or ABCG2-482T gene was introduced. In the drawing, “vector” represents MCF-7 cell with introduced the vector only, “T8” represents MCF-7 cell with introduced ABCG2-482T (MCF-7/T8), and “R7” represents MCF-7 cell with introduced ABCG2 of the present application (MCF-7/R7).

### DETAILED DESCRIPTION OF THE INVENTION

The present inventors have been studied hard to solve the above problems. Firstly, they established cell lines resistant to Compound-A. More specifically, these cell lines were obtained by cultivating mouse cell line LY, human cell line HCT116, and human cell line PC-13 under the presence of Compound-A for a long time. All of the obtained Compound-A resistant cell lines, i.e., LY/NR2, HCT116/NR1, and PC-13/NR13, and a spontaneous cell line HeLa #7 showed 50 to several thousand fold resistance to indolocarbazole compounds such as Compound-A, whereas only 20 fold or the like to other anticancer drugs such as camptothecin, adriamycin and mitoxantrone. Thus, the existence of a resistant mechanism specific to indolocarbazole compounds in these resistant cell lines was suggested. It was also speculated that a factor exists which decreases the intracellular concentration of indolocarbazole compounds and causes the resistance to the compounds in the resistant cell lines by finding decreased intracellular accumulation of indolocarbazole compounds such as Compound-A in the resistant cell lines.

Next, the present inventors compared the expressions of total known transcripts between the mouse cell line LY/NR2 resistant to Compound-A and the parent cell line LY by using DNA chips. As a result, a genomic sequence (ABCG2 of the present invention) having a high homology with the gene of ABCG2 sub-family which is a member of ABC transporter was found as a gene which showed the most increased expression level specific to the resistant cell line.

According to Northern analysis, it was found that the above gene was overexpressed not only in the mouse cell line LY/NR2, but also in all of the obtained human resistant cell lines. Therefore, in view of the fact that the ABCG2 gene of the present invention was expressed in all of the cell lines resistant to Compound-A and the previous finding that the aforementioned BCRP and the like are associated with the excretion of anti-cancer drugs, it was strongly suggested that the gene product of the present invention was a factor that confers the resistance to indolocarbazole compounds.

Then, the present inventors isolated a full-length cDNA of the present invention from human spontaneous resistant cell line HeLa #7 by RT-PCR method using primers designed based on the previous human ABCG2 sequence, determined the nucleotide sequence, and have found that the sequence of gene of the present invention was a novel one different from any other ABCG2 sequences previously reported. Namely, the gene of the present invention was a variant in which the codon sequence coding for threonine at amino acid residue 482 of the amino acid sequence reported as BCRP was changed to that coding for arginine by a single nucleotide substitution. Further, the present inventors determined the nucleotide sequence of ABCG2 which is obtained from cDNA derived from normal human tissues, i.e. placenta and kidney, wherein they focused attention on the part of the sequence different from the existing ABCG2, i.e. BCRP and MXR, sequences and have found out that the protein that has the same nucleotide sequence as that obtained from HeLa #7 cell line was expressed in normal human tissues.

The present inventors studied whether the gene of the present invention was capable of conferring resistance to indolocarbazole compounds on a cell. For the sake of study, a full length gene isolated from HeLa#7 was ligated with an expression vector and a stably transformed PC-13 cell line in which the gene was forcedly expressed was produced. As a result, the cell in which the ABCG2 gene of the present invention was forcedly expressed was approximately 10 to 20 fold resistant to indolocarbazole compounds. It was therefore proved that the gene of the present invention is a resistant factor to indolocarbazole compounds. At the same time, the present inventors have found that the cell expressing the gene of the present invention was not resistant to mitoxantrone and adriamycin in contrast to the previously reported cell line expressing the BCRP. Thus, it was proved that the gene of the present invention encoding a protein which has one amino acid replacement is a new type of gene having a novel activity.

Further, in order to clarify the relationship between one amino acid difference observed in the sequences of ABCG2 of the present invention compared with the sequence reported as BCRP (Doyle, A. et al., Proc. Natl. Acad. Sci. U.S.A. 95, 15665-15670 (1998) and WO99/40110) and the resistance to each drug, the present inventors introduced each of ABCG2 of the present invention and an amino acid variant of ABCG2 (ABCG2-482T) in which the codon of the 482nd amino acid was replaced to that of threonine as reported for BCRP, into MCF-7 cell and compared the characteristics of the cells.

As a result, the cell in which ABCG2-482T was introduced showed resistance to mitoxantrone and adriamycin in addition to Compound-B as an indolocarbazole compound, whereas the cell in which ABCG2 of the present invention was introduced showed strong resistance only to Compound-B. Thus, it was proved that the ABCG2 gene of the present invention shows the selective resistance to indolocarbazole compounds. There was also observed the same selectivity difference with respect to the accumulation of these compounds in the cells, and it was suggested that the selective resistance to indolocarbazole compounds caused by ABCG2 of the present invention is due to the substrate specific transport by this molecule. As a consequence of these observations, the distinct difference between the characteristics of ABCG2 as was previously reported BCRP and the ABCG2 of the present invention has been found out and it was shown that the difference was based on one amino acid substitution at codon 482.

Accordingly, in view of the above evidence, i.e. the fact that the ABCG2 gene of the present invention confers the resistance selective to indolocarbazole compounds on a cell, the present inventors have found that the gene and the protein of the present invention and fragments thereof or the antibodies thereagainst are applicable to detect the gene expression in a cancer patient and predict whether the cancer patient is resistant selective to indolocarbazole compounds or not. It was further found that the screening of compounds which regulates the expression of the gene or the activity of the gene product can be developed, and the application of the compounds to overcome the drug resistance.

The present invention relates to a novel drug resistance-associated gene and a product thereof. The nucleotide sequence of cDNA encoding a protein, “ABCG2 of the present invention” (human ABCG2), which has been isolated by the present inventors and which confers resistance to cancer chemotherapeutic drugs on a mammalian cell, is shown in SEQ ID NO:1, and the amino acid sequence of the protein, “ABCG2 of the present invention”, coded by the cDNA is shown in SEQ ID NO:2. The cDNA sequence of ABCG2 of the present invention has been discovered based on the screening of a gene that is highly expressed in a cell resistant to Compound-A which is an indolocarbazole compound, and has a two nucleotide difference compared with the sequence disclosed as BCRP among the ABCG2 sub-family which belongs to ABC transporter superfamily. Thus, the cDNA of ABCG2 of the present invention encodes a novel protein having an amino acid sequence different from the BCRP in one amino acid residue.

However, in the course of solving the problems of the present invention, it was confirmed that the degree of resistance to various drugs and the amount of intracellular drug accumulation of cell lines transfected with ABCG2 gene of the present invention were different from those of cell lines transfected with a gene disclosed as BCRP. In a cell line over-expressing ABCG2 or transfected with ABCG2 gene of the present invention, the resistance selective to indolocarbazole compounds was observed. Namely, it has been proved that the ABCG2 of the present invention is a novel drug resistance-associated gene encoding a protein having a different character from that of BCRP by substituting one amino acid residue thereof.

In the present invention, the phrase “mammalian cells” means cells obtained from a mammalian source, such as mammalian tissues, or external cell cultures of such cells.

In the present invention, the phrase “cancer chemotherapeutic drug” means a drug used for the purpose of treating cancers, and includes a synthetic compound, natural compound derived from plants or microorganisms, and semi-synthetic compound synthesized from a natural compound. In a preferred embodiment, the “cancer chemotherapeutic drug” refers to a compound represented by formula (I):

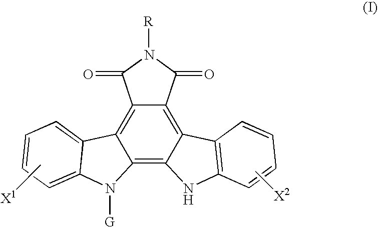

wherein X¹ and X² each independently represent a hydrogen atom, a halogen atom or a hydroxyl group;

R represents a hydrogen atom, amino, formylamino, or lower alkylamino which may be substituted with any one selected from the group consisting of one to three hydroxyl group(s), a pyridyl group optionally having substituent(s), and thienyl group optionally having substituent(s); and

G represents a pentose group, hexose group or derivative thereof which may be substituted with an amino group (hereinafter, generically called as indolocarbazole compound).

In a more preferred embodiment of the present invention, it refers to a compound represented by formula (I),

wherein X¹ and X² each independently represent a halogen atom or a hydroxyl group;

R represents a hydrogen atom, formylamino, or lower alkylamino which may be substituted with any one selected from the group consisting of one to three hydroxyl groups, a pyridyl group optionally having substituent(s), and thienyl group optionally having substituent(s); and

G represents a hexose group which may be substituted with an amino group.

The method for producing the indolocarbazole compounds have been described previously in, for example, European patent publication 0528030A1, U.S. Pat. Nos. 5,591,842, 5,668,271, and 5804564, WO95/30682, WO96/04293, WO98/07433, and JP Patent Kokai Publication No. JP-A-10-245390.

The phrase “resistance to cancer chemotherapeutic drugs” means that a mammalian cell shows the resistance to the therapeutic effect of cancer chemotherapeutic drugs. It means resistance of a cancer cell based on the enhanced efflux activity of cancer chemotherapeutic drugs out of the cancer cell, the mutations of the target enzyme in the cancer cell, the reduction of activity of drug activating systems for prodrugs in vivo, the enhancement of damage repair system of the cancer cell, and the like. In a preferred embodiment, the resistance is caused by the decrease of the intracellular drug concentration due to the enhanced efflux of cancer chemotherapeutic drugs out of a cancer cell.

The phrase “to confer the resistance to cancer chemotherapeutic drugs on a mammalian cell” means giving the enhanced activity to excrete cancer chemotherapeutic drugs out of a cell to a mammalian cell. In a preferred embodiment, it means that this enhanced activity to excrete cancer chemotherapeutic drugs out of a cell is imparted by an amplification of the ABCG2 gene of the present invention belonging to the ABCG2 sub-family included in ABC transporter superfamily (ATP binding cassette superfamily: Higgin, C. F. (1992) Annu. Rev. Cell. Biol. 8, 67-113), an increase of the transcript of the ABCG2 gene of the present invention, or an overexpression of the translated product of the ABCG2 gene of the present invention. In addition, the term “to confer the resistance to cancer chemotherapeutic drugs on a mammalian cell” means that a mammalian cell obtains the resistance to cancer chemotherapeutic drugs, due to the decrease of intracellular concentration of the cancer chemotherapeutic drugs in consequence of active efflux of the cancer chemotherapeutic drug incorporated into the mammalian cell by the function of ABCG2 gene product of the present invention.

The “ABCG2 protein of the present invention” includes a naturally occurring protein and also a recombinant protein prepared by recombinant DNA technologies. A naturally occurring protein can be prepared, for example, by subjecting tissue extract, such as placenta, which express the “ABCG2” protein of the present invention to affinity chromatography using an anti-ABCG2 antibody as described herein. In addition, other conventional purification and/or chromatography methods may be employed. A recombinant protein can be prepared by cultivating a cell which is transformed with a DNA encoding human “ABCG2” protein of the present invention and further purified, if desired, as is described below.

The human ABCG2 protein of the present invention (SEQ ID NO: 2) can be modified using known methods to prepare a modified protein such that the modified protein has an equivalent function of the protein of SEQ ID NO:2, that is, a binding activity to or a transporting activity of indolocarbazole compounds. In a preferred embodiment, the modified protein also has the selective binding and transporting activity for the indolocarbazole compounds. In another embodiment, the modification or mutation of the amino acid sequence may occur naturally and thus is included in the present invention, preferably such a naturally modified protein will have the activity of SEQ ID NO:2 as described herein.

Methods for modifying amino acid residues include, for example, Kunkel method (Kunkel, T. A. et al., Methods Enzymol. 154,367-382 (1987)), Double primer method (Zoller, M. J. and Smith, M., Methods Enzymol. 154, 329-350 (1987)), cassette mutagenesis method (Wells, et al., Gene 34, 315-23 (1985)), mega-primer method (Shaker, G. and Sommer, S. S., Biotechniques 8, 404-407 (1990)). The number and position of mutated amino acids of a protein are not limited so far as the function of the protein is maintained, however, the number of mutated amino acid(s) in a functionally equivalent protein is generally within 10% of total amino acids, preferably within 10 amino acids, and more preferably within 3 amino acids (for example, 1 amino acid) and the amino acid at position 482 of the protein of the present invention is arginine.

In one embodiment of the invention, the modified protein will have at least 70%, 80%, 85%, 90%, 95%, 97%, 98%, and 99% homology to SEQ ID NO:2, homology being determined using the BLAST algorithm (Altschul S F J Mol Biol Oct. 5, 1990;215(3):403-10). The homologous modified protein has substantially the same activity and selectivity as described herein for the protein containing SEQ ID NO:2. In a preferred embodiment, the homologous modified protein has the ATP binding region along with the arginine residue corresponding to position 482 in SEQ ID NO:2.

The present invention also includes a partial peptide of the aforementioned human ABCG2 protein of the present invention. For example, the partial peptide includes but is not limited to a partial peptide having a ATP binding region of ABCG2 protein of the present invention (amino acid residues 61 to 270), a partial peptide having an arginine at the 482nd residue in the amino acid sequence of the protein of the present invention, a partial peptide having a binding activity with indolocarbazole compounds, a partial peptide having an activity to excrete indolocarbazole compounds out of cells when it is expressed on the surface of a cell. These partial peptides can be used, as is the case with the ABCG2 protein of the present invention, for example, for preparing an antibody, screening a candidate compound of the drug as described below, or screening a compound that enhances the therapeutic efficacy of indolocarbazole compounds. Moreover, a partial peptide which has a binding activity to indolocarbazole compounds, without any drug excreting activity out of a cell, may be a competitive inhibitor of the ABCG2 protein of the present invention. Such partial peptides of the present invention may have at least 15 amino acid residues, preferably 20 amino acid residues or more in chain length. The partial peptides of the present invention can be prepared, for example, by a genetic engineering technique, the peptide synthesis method known in the art, or the digestion of the protein of the present invention with appropriate peptidases.

The present invention also relates to a polynucleotide encoding the protein of the present invention or partial peptides thereof. As used herein, the term “polynucleotide” generally refers to both polyribonucleotide and polydeoxyribonucleotide, which can be either a unmodified RNA or DNA, and either a modified RNA or DNA. There is enumerated, for example, DNA, cDNA, genomic DNA, mRNA, unprocessed RNA and a fragment thereof, without particular restriction of its length.

The phrase “a polynucleotide encoding a protein or partial peptide thereof” refers to a polynucleotide having any nucleotide sequence based on the degeneracy of the genetic code, provided that each sequence can encode the ABCG2 protein of the present invention or partial peptide thereof. As such polynucleotides, for example, there are provided a DNA encoding the ABCG2 protein of the present invention or partial peptide thereof and an RNA such as mRNA. These may be double stranded or single stranded. In the case of double stranded, it may be double stranded DNA, double stranded RNA or DNA:RNA hybrid. The DNA encoding the ABCG2 protein of the present invention or partial peptide thereof includes a genomic DNA, genomic DNA library, cDNA derived from the aforementioned cells and tissues, and synthetic DNA.

The cDNA encoding the ABCG2 protein of the present invention can be screened, for example, by labeling the cDNA represented in SEQ ID NO: 1 or a fragment thereof, complementary RNA thereto, or synthetic oligonucleotides including a portion of the cDNA sequence, with ³²P or the like, and hybridizing them with a cDNA library derived from a tissue expressing the ABCG2 protein of the present invention (e.g., placenta). Alternatively, the cDNA can be cloned by synthesizing oligonucleotides corresponding to the cDNA sequence, and amplifying template cDNA derived from the appropriate tissue (e.g., placenta) by the polymerase chain reaction. The genomic DNA can be screened by, for example, labeling the cDNA represented in SEQ ID NO: 1 or a fragment thereof, a complementary RNA thereto, or a synthetic oligonucleotide including a portion of the cDNA sequence, with ³²P or the like, and then hybridizing to the genomic DNA library. Alternatively, the genomic DNA can be cloned by synthesizing oligonucleotides corresponding to the cDNA sequence and amplifying the genomic DNA as the template by polymerase chain reaction. On the other hand, a synthetic DNA can be prepared by, for example, chemically synthesizing oligonucleotides having a partial sequence of the cDNA represented in SEQ ID NO: 1, annealing them to form double strand, and ligating them with DNA ligase (Khorana, H. G. et al., J. Biol. Chem. 251, 565-570 (1976); Goeddel D. V. et al., Proc Natl. Acad. Sci. U.S.A. 76, 106-10 (1979)).

In another aspect of the present invention, polynucleotides that have at least 70%, 80%, 85%, 90%, 95%, 97%, 98%, and 99% homology to SEQ ID NO:1, homology being determined using the BLAST algorithm (Altschul S F J Mol Biol Oct. 5, 1990;215(3):403-10). The homologous polynucleotide encodes a protein having substantially the same activity and selectivity as described herein for the protein containing SEQ ID NO:2. In a preferred embodiment, the homologous polynucleotide encodes a protein having the ATP binding region along with the arginine residue corresponding to position 482 in SEQ ID NO:2.

The polynucleotide of the present invention can be also used for detecting a mammalian cell having resistance to the compounds represented in formula (I). The detection method includes extraction of mRNA from a mammalian cell as a sample by a general method and measuring the amount of ABCG2 mRNA by any method such as northern hybridization using the polynucleotide of the present invention as a probe, or performing RT-PCR using a primer set that can hybridize to the polynucleotide sequence of the present invention of the extracted RNA. In this case, the overexpression of the mRNA may be an indication to detect a mammalian cell having resistance to the compound represented in formula (I).

In order to detect the mRNA or genomic DNA characteristic to the ABCG2 protein of the present invention, a polymorphism corresponding to the codon 482 encoding arginine residue of the ABCG2 protein can be detected. As a method of detecting such a polymorphism, for example, the mRNA extracted from a selected cancer cell may be hybridized with DNA chips and the like on which polynucleotides consisting of 15 to 100 contiguous nucleotides of the sequences of SEQ ID NO:1 and comprising a nucleotide at position 1489 (G, a polymorphic site) of the DNA represented in SEQ ID NO: 1, or complement thereof, is immobilized to detect the nucleotide sequence of the aforementioned polymorphic site.

The DNA of the present invention is useful for the production of the recombinant protein. Namely, it is possible to prepare the human ABCG2 protein of the present invention as a recombinant protein by constructing an appropriate expression vector with the inserted DNA encoding the ABCG2 protein of the present invention (e.g., the cDNA represented in SEQ ID NO: 1), cultivating the transformant obtained by introducing the vector into an appropriate cell, and purifying the expressed protein.

In another embodiment, the present invention relates to a recombinant vector comprising a polynucleotide of the present invention. The phrase “recombinant vector” refers to a DNA in which an exogenous DNA is incorporated and which can replicate in the host cell. These are prepared by modifying plasmids, phages or viruses which are all self-replicative. As a plasmid and the like which is used for preparing a recombinant vector of the present invention, there is no particular limitation if it can stably hold the inserted polynucleotide. For example, in a case where the host is Escherichia coli, a plasmid vector such as pET-3 (Rosenberg, A. H., et al., Gene 56,125-35 (1987)), pGEX-1 (Smith, D. B. and Johnson, K. S., Gene 67, 31-40 (1988)) is used. In a case where the host is a fission yeast, Schizosaccharomyces pombe, a plasmid vector such as pESP-1 (Lu, Q. et al., Gene 200, 135-144 (1997)) is used. In a case where the host is an insect cell, a baculovirus vector pBacPAK8/9 (Clontech) and the like are used. On the other hand, in a case where the host is a mammalian cell such as a Chinese hamster ovary (CHO) cell and a human HeLa cells, vectors such as pMSG (Amersham Pharmacia Biotech.), pcDNA (Invitrogen Corporation) and the like are used.

In still another embodiment, the present invention relates to a transformant comprising the recombinant vector of the present invention. The term “transformant” refers to a cell in which the exogenous DNA is incorporated by a recombinant vector. The host cell in which the recombinant vector of the present invention is introduced may be a prokaryotic cell or a eucaryotic cell, and includes any host cell which can be used for the object of the present invention, such as bacterial cell, yeast cell, insect cell, and mammalian cell. The specific methods for introducing a recombinant vector into the host cell, and obtaining the transformants are as follows. Transformation of E. coli. is performed by the method of Hanahan (Hanahan, D., J. Mol. Biol. 166, 557-580 (1983)), the electroporation (Dower, W. J. et al., Nucl. Acids Res. 16, 6127-6145 (1988)) and the like. Transformation of yeast is performed by, for example, spheroplast method (Beach, D. and Nurse, P., Nature 290, 140 (1981)), lithium acetate method (Okazaki, K. et al., Nucleic Acids Res. 18, 6485-6489 (1990)). Transformation of an insect cell is performed by, for example, a method described in Bio/Technology, 6, 47-55 (1980). Transfection of a recombinant DNA into a mammalian cell is performed by calcium phosphate method (Graham, F. L. and van der Eb, A. J., Virology 52, 456-467 (1973)), DEAE-dextran method (Sussman, D. J. and Milman, G., Mol. Cell. Biol. 4,1641-1643 (1984)), lipofection method (Felgner, P. L. et al., Proc. Natl. Acad. Sci. USA 84, 7413-7417 (1987)), electroporation (Neumann, E. et al., EMBO J. 1, 841-845 (1982)) and the like.

In a further embodiment, the present invention relates to a method for producing the protein of the present invention or partial peptides thereof by using the transformant of the present invention. A recombinant protein expressed by the transformant can be isolated from the transformant or culture supernatant thereof according to a standard method of the art. The standard method in this context includes ammonium sulfate precipitation, column chromatography (for example, ion exchange, gel filtration, and affinity chromatography) and electrophoresis. In addition, the recombinat protein can be purified by, for example, synthesizing it as a fusion protein attached at N-terminus with histidine residue tag sequence or glutathione S-transferase (GST) and the like, and binding it to metal chelating resin or GST affinity resin (Smith, M. C. et al., J. Biol. Chem. 263, 7211-7215 (1988)), respectively. When pESP-1 is used as a vector, for example, the protein of interest is synthesized as a fusion protein with glutathione S-transferase (GST), therefore, the recombinant protein can be purified by binding it to GST affinity resin. The fusion protein may be cleaved with thrombin, blood coagulation factor Xa, and such, to liberate the protein of interest from the fusion protein.

In a still further embodiment of the present invention, an antibody which binds to the human ABCG2 protein of the present invention is provided. In one embodiment, the antibody specifically binds to that epitope responsible for the selectivity of the ABCG-2 protein for indolocarbazole compounds. The antibody that binds to the protein of the present invention can be prepared according to the method known to the skilled person in the art (e.g. refer to “New experimental course of biochemistry 1, Protein 1,389-406, Tokyo Kagaku Dojin”). The preparation of polyclonal antibody is performed, for example, as follows. To an immunocompetent animal such as rabbit, guinea pig, mouse and chicken is injected the ABCG2 protein of the present invention or partial peptide thereof. The injection may be accompanied by the adjuvant (FIA or FCA) which promotes the antibody production. Administration in usually carried out every several weeks. The titer of antibodies can be elevated by multiple immunizations. After the final immunization, antiserum is obtained by collecting blood from the immunized animal. The polyclonal antibody can be prepared from this antiserum by, for example, fractionation with ammonium sulfate precipitation or anion exchange chromatography, or by affinity purification using Protein A or immobilized antigen. On the other hand, a monoclonal antibody is prepared, for example, as follows. The ABCG2 protein of the present invention or partial peptide thereof is immunized to an immunocompetent animal as described above, and after the final immunization, spleen or lymph node is harvested from the immunized animal. A hybridoma cell is prepared by the cell fusion of the antibody producing cell which is contained in this spleen or lymph node and a myeloma cell using polyethylene glycol and the like. By screening and cultivating the target hybridoma cell, the monoclonal antibody can be prepared from the culture supernatant. The monoclonal antibody can be purified by, for example, fractionation with ammonium sulfate precipitation or anion exchange chromatography, or by affinity purification using Protein A or immobilized antigen. The antibody thus prepared is used for affinity purification of the ABCG2 protein of the present invention And also, it may be used for detecting the amount of the expression of the ABCG2 protein of the present invention as well. The detection of the expression amount of human ABCG2 protein in a mammalian cell by the antibody makes it possible to determine the resistance of the mammalian cell to the compound represented in formula (I), wherein the resistance is indicated by the overexpression of the protein. In addition, the detection of the human ABCG2 protein in a cancer cell or cancer patient by the antibody can be used for diagnosing the disease or drug resistance caused by the overexpression of the protein. This antibody can be further used for the antibody therapy against those diseases or drug resistance.

It is preferable to use humanized antibodies or human antibodies for the antibody therapy. A humanized antibody, for example, a mouse-human chimeric antibody can be prepared by, for example, isolating the gene encoding the antibody against the ABCG2 protein of the present invention from antibody-producing mouse cells, replacing the constant region on the H chain of the antibody with the human IgE, and introducing into a mouse myeloma cell J558L, (Neuberger, M. S. et al., Nature 314, 268-270 (1985)). Alternatively, human antibody can be prepared by immunizing mice whose immune systems have been replaced with that of humans with the ABCG2 protein of the present invention.

In an embodiment, the present invention relates to an antisense nucleotide suppresses the expression of the protein of the present invention. The “antisense nucleotide(s)” of the present invention includes a sequence which specifically hybridizes to any region of mRNA encoding the ABCG2 protein of the present invention or partial peptide thereof, or any region of 5′ or 3′ untranslated region of the mRNA, and is capable of preventing the translation of the mRNA. The antisense nucleotide(s) can contain a sequence which is capable of specifically hybridizing with any region of the cDNA sequence encoding the ABCG2 protein of the present invention and partial peptide thereof. Specifically, the antisense nucleotide(s) can be designed based on the nucleotide sequence represented in SEQ ID NO:1. It can consist of a complementary sequence of any sequence of coding region or untranslated region of the represented sequence. The antisense nucleotide of the present invention may be RNA, DNA or modified nucleic acid (RNA, DNA), and may contain modified sugars, bases or linkages. The specific examples of the modified nucleic acid includes but is not limited to a sulfurized derivative, thiophosphate derivative and the like.

The inhibitory activity of the antisense nucleotide can be evaluated by using the transformant of the present invention, in vivo or in vitro gene expression system of an ABCG2 protein, or, an in vivo or in vitro translation system of an ABCG2 protein. The antisense nucleotide can be placed in the cell through any number of ways known per se. For example, the antisense nucleotide can be delivered in the specialized systems such as liposome or microsphere, or may have attached moieties. Such attached moieties include polycationic substance as polylysine which act as charge neutralizers of the phosphate backbone, or hydrophobic substance as lipids (for example, phospholipids, cholesterols) that enhance interaction with cell membrane and increase uptake of nucleic acid. It is applicable to gene therapy as well as cultured cell. As well as by the introduction of the oligonucleotide itself from the outside as described above, the antisense nucleotide(s) can be also performed through the mediation of a vector and the like which is capable of expressing the antisense nucleotide in vivo. In the Japanese patent Kohyo publication JP-A-11-511965, it has been shown that an antisense oligonucleotide which suppresses the expression of human genes MDR1 and MRP encoding multi-drug resistance associated proteins enhanced the sensitivity of a multi-drug resistant cell to the drugs. According to the method described in this prior art, the sequence of the antisense nucleotides of the present invention can be designed by selecting an appropriate target region. The antisense nucleotides of the present invention can be used for enhancing the sensitivity to an indolocarbazole compound by administering it to a drug-resistant cancer cell or cancer patient in which the ABCG2 protein of the present invention is overexpressed.

In another embodiment of the present invention, there is provided a method for predicting the resistance of a patient to cancer chemotherapeutic drugs, wherein the resistance is indicated by the expression of the protein of the present invention. The term “expression” in the phrase “indicated by the expression” includes both meanings of the process transcribed from DNA to mRNA and the process further translated to a polypeptide or protein.

The term “indicated by the expression of the protein” means that the resistance to cancer chemotherapeutic drugs is indicated by comparing the amount of mRNA of the protein or the amount of the protein (polypeptide) itself in the sample with those of surrounding normal tissues or culture cells of known drug resistance to assess the amount of the expressed protein. In a specific embodiment of the present invention, the comparison of the amount of mRNA is performed by extracting RNA by routine methods from a mammalian cell, cancer cell of a patient, tissue preparation, tissue sample or the like, and assaying by northern hybridization, as is shown in Example 4, with DNA fragment comprising a partial sequence of the polynucleotide encoding the ABCG2 protein of the present invention as a probe. Alternatively, the amount of mRNA of the protein is determined by performing RT-PCR according to the literatures (Noonan, K. E., et al., Proc. Natl. Acad. Sci. USA (1990) 87, 7160-4 and Futscher, B. W., et al., Anal Biochem (1993) 213,414-21) by using the extracted RNA and a primer set which can hybridize to the polynucleotide sequence encoding the ABCG2 protein of the present invention. The methods which measure the amount of mRNA, however, are not limited to these methods.

In the case of measuring and comparing the amount of the protein (polypeptide) itself, the method includes but is not limited to the comparison to the control by the immunohistochemical staining of a mammalian cell, cancer cell of a patient or tissue section, according to the method of the literature (Beck, W. T., et al., Cancer Res. (1996) 56, 3010-20) with the aforementioned “antibody”, or by the western blotting using a standard method with respect to the protein extracts of the sample.

When the protein is observed to be overexpressed in the sample compared to controls by any method described above or another, the cell or the patient derived from the sample is decided to be resistant to cancer chemotherapeutic drugs.

The present invention also includes a method for screening a compound which inhibits the function of the ABCG2 protein of the present invention. This is a method which searches any one of substances as follows. (i) a substance which inhibits the binding of the ABCG2 protein of the present invention to a substrate compound thereof. (ii) a substance which inhibits the activation of ATPase activity of the ABCG2 protein of the present invention by binding with its substrate compound. (iii) a substance which inhibits the membrane transport of a substrate compound by the ABCG2 protein of the present invention. (iv) a substance which enhances the cytotoxicity of a substrate compound of the ABCG2 protein of the present invention.

The screening method of the present invention which searches the substance (i) comprises the steps: (a) contacting the ABCG2 protein of the present invention or partial peptide thereof with its substrate compound in the presence of a candidate compound, and measuring the binding activity between the protein or partial peptide thereof and the substrate compound, and (b) comparing the binding activity detected in the step (a) with that in the absence of the candidate compound and selecting a compound which suppresses the binding activity between the ABCG2 protein of the present invention or partial peptide thereof and the substrate compound. A candidate compound includes but is not limited to a protein, peptide, non-peptide compound, artificially synthesized compound, tissue extract, cell extract, and/or serum. The ABCG2 protein of the present invention or partial peptide thereof used for screening does not limited to its purified state but can be various forms, for example, in a form of bound to an affinity column, in a form of a membrane vesicle of a desired cell (including a transformant genetically engineered to express the protein) in which the protein or partial peptide thereof is expressed in the membrane (which can be prepared according to the method of Leier I. et al., Journal of Biological Chemistry 269 (45): 27807-10, 1994), or in a form of a reconstituted vesicle of the purified protein or partial peptide on a liposome (which can be prepared according to the method of Anbudkar, S. V. et al., Proc. Natl. Acad. Sci. USA (1992) 89: 8472-8476). The substrate compounds used for the screening are not limited to certain substances, but are preferably indolocarbazole compounds, for example, Compound-A, which are used by labeling as necessary. The label includes but is not limited to, for example, a radiolabel, fluorescence label, photoaffinity label. The binding activity of the ABCG2 protein of the present invention to the substrate compound can be detected through the labeling of the compound bound to the ABCG2 protein of the present invention (for example, by measuring radioactivity or fluorescence intensity). In case of a photoaffinity label, the binding activity can be also detected according to the method of the literature (Cornwell M M. et al., Proc. Natl. Acad. Sci. USA. 83:3847-50, 1986) by isolating a covalently bonded complex between the protein or partial peptide thereof and a photoaffinitylabeled compound using SDS polyacrylamide gel and the like, and then measuring the radioactivity of the photoaffinitylabeled compound by autoradiography. As a result of the detection, it is determined that the candidate compound has the inhibitory activity of the binding between the ABCG2 protein of the present invention and the substrate compound, when the binding activity in the presence of the candidate compound is lower than that in the absence of the candidate compound (control experiment).

The screening method of the present invention which searches the substance (ii) comprises the steps: (a) measuring ATPase activity when the ABCG2 protein of the present invention or partial peptide thereof is contacted with a substrate compound in the presence of a candidate compound, and (b) comparing the ATPase activity detected in the step (a) With that in the absence of the candidate compound, and selecting a compound which suppresses the ATPase activity of the ABCG2 protein of the present invention by binding of the substrate compound. The candidate compound includes but is not limited to a protein, peptide, non-peptide compound, artificially synthesized compound, tissue extract, cell extract, and/or serum. The ABCG2 protein of the present invention or partial peptide thereof which is used for the screening method can be, for example, in a form of a membrane vesicle of a desired cell (including a transformant genetically engineered to express the protein), in which the protein or partial peptide thereof is expressed in the membrane or in a form of reconstituted form of the purified protein or partial peptide on a liposome. The substrate compounds used for the screening is not limited to certain compounds but are preferably indolocarbazole compounds, for example, Compound-A. The ATPase activity can be measured by a standard method, for example, a colorimetric amounts of inorganic phosphate produced by hydrolysis of ATP (Adam B. Shapiro et al., Journal of Biological Chemistry, 269:3745-3754, 1994). It is decided that the candidate compound has the inhibitory activity of the activation of ATPase activity of the ABCG2 protein of the present invention by the substrate compound, when the ATPase activity in the presence of the candidate compound is lower than that in the absence of the candidate compound (control experiment).

The screening method of the present invention by searching the substance (iii) is classified broadly into three methods.

The first method uses a membrane vesicle of a desired cell (including a transformant genetically engineered to express the protein) in which the ABCG2 protein of the present invention or partial peptide thereof is expressed in the membrane, or through a reconstituted vesicle of the purified protein or partial peptide on a liposome, and comprises the steps: (a) contacting the vesicle with a substrate compound of the protein in the presence of a candidate compound, and then, measuring the amount of the substrate compound which is transported into the vesicle and (b) comparing the amount of the substrate compound transported into the vesicle and measured in the step (a) with that in the absence of the candidate compound and selecting a compound which inhibits the membrane transporting activity of the substrate compound by the ABCG2 protein of the present invention or partial peptide thereof. Specifically, the method can be performed by, for example, a method according to the description in J. Biol. Chem. 269, 27807-10 (1994). The substrate compounds used for the screening are not limited to certain compounds but are preferably indolocarbazole compounds, for example, Compound-A, which are used by labeling as necessary. The label includes but is not limited to, for example, a radiolabel, fluorescence label, photoaffinity label. The amount of the substrate compound transported into the vesicle is detected by these labels (for example, by measuring radioactivity or fluorescence intensity). As a result of the detection, it is decided that the candidate compound has the inhibitory activity of the membrane transporting activity of the substrate compound by the ABCG2 protein of the present invention or partial peptide thereof, when the amount of the intravesical substrate compound in the presence of the candidate compound is lower than that in the absence of the candidate compound (control experiment).

The second method uses a desired cell (including a transformant genetically engineered to express the protein) in which the ABCG2 protein of the present invention or partial peptide thereof is expressed in (through) the membrane, and comprises the steps: (a) measuring the amount of a substrate compound after contacting the cell with the substrate compound of the protein in the presence of a candidate compound and (b) comparing the amount of the substrate compound accumulated in the cell and measured in the step (a) with that in the absence of the candidate compound and selecting a compound which inhibits the membrane transporting (excreting) activity of the substrate compound out of the cell by the ABCG2 protein of the present invention or partial peptide thereof.

The third method uses a desired cell (including a transformant genetically engineered to express the protein) in which the ABCG2 protein of the present invention or partial peptide thereof is expressed in(through) the membrane, and comprises the steps: (a) contacting the cell with the substrate compound of the protein to accumulate the substrate compound in the cell, and (b) measuring an amount of the residual substrate compound in the cell after cultivation of certain period of the cell obtained in the step (a) in the presence of a candidate compound, and (c) comparing the amount of the substrate compound accumulated in the cell and detected in the step (b) with that in the absence of the candidate compound, and then selecting a compound which decrease the membrane transporting (excreting) activity of the substrate compound out of the cell by the ABCG2 protein of the present invention or partial peptide thereof. The second and third methods can be performed by a method according to the description in Bruin, M. et al., Cancer. Let. 146, 117-26 (1999). In each of the second and third method, the used substrate compounds are not limited to certain compounds, but includes a fluorescent substance which can penetrate into a cell and can be transported by the ABCG2 protein of the present invention, or indolocarbazole compounds, for example, Compound-A, which are used by labeled as necessary. As a label, for example, a radiolabel, fluorescence label, photoaffinitylabel and the like are enumerated. The amount of the substrate compound accumulated or remained in a cell is detected by these labels (for example, by measuring radioactivity in case of a radiolabel, or fluorescence intensity by flow cytometry, fluorescence microscope, fluorophotometer and the like in case of a fluorescence label). As a result of the detection, it is decided that the candidate compound has an inhibitory activity of the membrane transporting activity of the substrate compound by the ABCG2 protein of the present invention or partial peptide thereof, when the amount of the intracellular accumulation or residue of the substrate compound in the presence of the candidate compound is higher(more) than that in the absence of the candidate compound (control experiment). In any of the first, second and third screening methods, the candidate compound includes but is not limited to a protein, peptide, non-peptide compound, artificially synthesized compound, tissue extracts, cell extracts, and/or serum.

The screening method of the present invention by searching the substance (iv) uses a desired cell (including a transformant genetically engineered to express the protein) in which the ABCG2 protein of the present invention or partial peptide thereof is expressed in(through) the membrane, and comprises the steps: (a) cultivating the cell with a substrate compound of the protein for a constant time in the presence of a candidate compound and measuring the number of viable cells, and (b) comparing the viable cell number measured in the step (a) with that in the absence of the candidate compound, and then selecting a compound which enhances the cytotoxicity of the substrate compound. The candidate compound includes but is not limited to a protein, peptide, non-peptide compound, artificially synthesized compound, tissue extract, cell extract, and/or serum and the like. The substrate compounds used for the screening are not limited to certain compounds but are preferably indolocarbazole compounds, for example, Compound-A, which have a cytotoxic effect. It is well known to those of the skilled person in the art that the measurement of the number of viable cells can be replaced by the measurement of the amount of the protein or that of the activity of mitochondrial reducing enzyme.

Inhibitors to be obtained by the screening method of the present invention as described above can confer suppressed resistance to drugs on a cell and patient that are resistant to the drugs because of efflux of the drugs out of the cell caused by the ABCG2 protein of the present invention. Namely, the inhibitors are useful as drug-sensitivity enhancing agents. Pharmaceutical compositions comprising these compounds may be useful for treatment of diseases associated with membrane transport systems mediated by the ABCG2 protein of the present invention. These compositions can include any acceptable pharmaceutical carrier for administration by a suitable mode of delivery, such as oral or parenterally. In one embodiment, the compositions would be formulated to provide slow release or time-delayed release of the inhibitor compound or composition. Furthermore, as more than one inhibitor may be useful, the present invention provides administering two or more inhibitor compounds together or separately.

Inhibitors obtained by the screening method of the present invention are compounds to inhibit the activity of the ABCG2 protein of the present invention. To be specific, the inhibitor binds with high affinity to the ABCG2 protein and inhibits the molecule from transporting the substrates thereof to the outside competitively or non-competitively. The inhibitor includes a peptide, protein, non-peptide compound, synthesized compound, fermentation product. These compounds may be novel compounds or known ones. These compounds can be administered to a cell expressing the ABCG2 protein together with an anticancer agent which is a substrate for the ABCG2 protein, to enhance the effect of the anticancer agent. This method is particularly useful for treatment for a cancer that aquired anticancer drug resistance through expressing the ABCG2 protein, and thus the inhibitory compound can be a pharmaceutical agent effective in overcoming the drug-resistance.

### EXAMPLES

The present invention is explained in more detail by reference to the following examples and reference examples, however, these examples do not restrict the scope of the present invention.

### Example 1

### Establishment of Cell Lines Resistant to Compound-A

To establish Compound-A-resistant cell lines from different cell lines, a total of three cell lines: the mouse fibroblast cell line LY, human lung cancer cell line PC-13, and human colon carcinoma cell line HCT116 were cultivated for a long time in the presence of Compound-A as follows.

### Example 1-1

### Establishment of Compound-A-Resistant Mouse Fibroblast Cell Line LY/NR1:

LY cells were cultivated in the presence of 0.1 μM Compound-A for 2 weeks, then in the presence of 0.3 μM for 3 weeks. A colony that grew under these conditions was isolated using a cloning ring (Asahi Techno Glass Corporation) and designated as LY/NR1.

### Example 1-2

### Establishment of Compound-A-Resistant Mouse Fibroblast Cell Line LY/NR2

LY cells were cultivated in the presence of 0.1 μM Compound-A for 2 weeks, and then in the presence of 0.3 μM for 5 weeks. An emerging colony was isolated as described above and designated as LY/NR2.

### Example 1-3

### Establishment of Compound-A-Resistant Human Cell Line PC-13/NR13

A Compound-A-resistant PC-13X13 cell line isolated after culture of PC-13 cells in the presence of 1.1 μM Compound-A for 5 weeks was cultured in the presence of 20 μM Compound-A for 4 weeks. An emerging colony was isolated and designated as PC-13/NR13.

### Example 1-4

### Establishment of Compound-A-Resistant Human Cell Line HCT116/NR1

A Compound-A-resistant HCT116X13 cell line isolated after culture of the HCT116 cells in the presence of 1.1 μM Compound-A for 5 weeks was cultured in the presence of 20 μM Compound-A for 4 weeks. An emerging colony was isolated and designated as HCT116/NR1.

### Example 1-5

### Selection of a Compound-A-Resistant Spontaneous Cell Line

A single clone was obtained by limiting dilution method from endocervical carcinoma cells, HeLa cells, which were spontaneous resistant to Compound-A, and designated as HeLa#7 cell line. The existing HeLaS3 cell line which is derived from the same original HeLa cells, was used as a Compound-A-sensitive cell for comparative experiments to the HeLa#7.

### Example 2

### Measurement of Drug Sensitivity of the Compound-A-Resistant Cell Lines to Various Anticancer Drugs

The sensitivities of the established Compound-A-resistant cell lines to Compound-A, Compound-B, and other anti-cancer drugs were measured using Sulforhodamine B dye-staining (SRB) method as follows.

Test cells in the logarithmic growth phase were-dispensed into wells of 96-well plate at 1×10³ cells/well. After subculture for 24 hours in a CO₂ incubator, a serial diluted agent was added to each well. After further incubation for 72 hours, the cells were fixed with trichloroacetic acid (TCA) and the proteins in the cells were stained by 0.4% sulforhodamine B solution. The samples were eluted by 10 mM Tris-HCl for 1 hour, and each well was measured at 560 nm with a 450 nm reference, by a SPECTRAmax 250 plate reader (Molecular Devices Corp.). In this specification, IC₅₀ was defined as an agent concentration to inhibit the growth of a cell line to 50%, and Relative Resistance was determined by dividing an IC₅₀ value of an agent to a resistant cell line by an IC₅₀ value of the agent to its parental cell line.

The measurement revealed that all of the Compound-A-resistant human cell lines displayed more than 100-fold resistance to both Compound-A and Compound-B as compared with their parental cell lines (Table 1). The mouse LY/NR1 cell line showed moderate resistance to Compound-A and Compound-B, the Relative Resistance of which were 12 and 17, respectively. The mouse LY/NR2 cell line displayed high resistance to Compound-A and Compound-B, the Relative Resistance of which were 64 and 210, respectively (Table 2).

TABLE 1
Sensitivities of human Compound-A resistant cell lines to various anti-cancer drugs

| | HCT116 | HCT116/NR1 | | PC13 | PC13/NR13 | | HeLaS3 | HeLa #7 | |
|---|---|---|---|---|---|---|---|---|---|
| Drug | IC50 (μM) | IC50 (μM) | RR | IC50 (μM) | IC50 (μM) | RR | IC50 (μM) | IC50 (μM) | RR |
| Compound-A | 0.13 | 300 | 2400 | 0.23 | >1000 | >4300 | 0.89 | 290 | 330 |
| Compound-B | 0.0034 | 0.78 | 230 | 0.013 | 1.8 | 140 | 0.015 | 200 | 13000 |
| Camptothecin | 0.0094 | 0.017 | 1.8 | 0.038 | 0.029 | 0.76 | 0.014 | 0.038 | 2.7 |
| Topotecan | 0.034 | 0.14 | 4.1 | 0.075 | 1.1 | 15 | 0.063 | 0.36 | 5.7 |
| Etoposide | 1.1 | 3.4 | 3.1 | 1.2 | 3.0 | 2.5 | 1.2 | 0.93 | 0.79 |
| Doxorubicin | 0.025 | 0.059 | 2.3 | 0.028 | 0.070 | 2.5 | 0.035 | 0.029 | 0.81 |
| Vincristine | 0.0020 | 0.0093 | 4.7 | 0.013 | 0.0070 | 0.56 | 0.00070 | 0.0069 | 9.9 |
| Paclitaxel | 0.0011 | 0.0021 | 1.9 | 0.0020 | 0.0018 | 0.92 | 0.0010 | 0.0042 | 4.2 |
| Mitoxantrone | 0.0070 | 0.067 | 9.6 | 0.033 | 0.16 | 4.9 | 0.0044 | 0.027 | 6.1 |

RR: Relative Resistance

TABLE 2
Sensitivities of mouse Compound-A resistant cell
lines to various anti-cancer drugs

| | LY | LY/NR1 | | LY/NR2 | |
|---|---|---|---|---|---|
| Drug | IC50 (μM) | IC50 (μM) | RR | IC50 (μM) | RR |
| Compound-A | 0.12 | 1.4 | 12 | 7.7 | 64 |
| Compound-B | 0.0017 | 0.029 | 17 | 0.36 | 210 |
| Camptothecin | 0.046 | 0.058 | 1.3 | 0.38 | 8.2 |
| Topotecan | 0.069 | 0.25 | 1.8 | 2.9 | 20 |
| Etoposide | 0.28 | 0.52 | 2.1 | 2.0 | 4.7 |
| Doxorubicin | 0.043 | 0.067 | 1.6 | 0.19 | 4.5 |
| Vincristine | 0.015 | 0.011 | 0.77 | 0.025 | 1.7 |
| Paclitaxel | 0.041 | 0.057 | 1.4 | 0.081 | 2.0 |
| Mitoxantrone | 0.0033 | 0.0071 | 1.5 | 0.057 | 17 |

RR: Relative Resistance

### Example 3

### Example 3-1

### Preparation of RNA from Cell Lines LY, LY/NR1, and LY/NR2

RNA from each of the cell lines LY, LY/NR1, and LY/NR2 was prepared by the following method. Approximately 1×10⁷ of cells were detached from the culture dish by trypsinization and collected by centrifugation. The cells were then homogenized using a QIAshredder (QIAGEN Inc.) and total RNA was isolated using an RNeasy total RNA isolation kit (QIAGEN Inc.).

### Example 3-2

### Preparation of cRNA

A complementary DNA was synthesized from 32 μg of each total RNA fraction supplemented with a primer, T7-(dT)₂₄ (SEQ ID NO: 3: GGCCAGTGMTTGTMTACGACTCACTATAGGGAGGCGG-(dT)₂₄), using SuperScript II Reverse Transcriptase (BRL) and a supplied buffer. Then the double stranded DNA was synthesized by adding E. coli DNA Ligase (BRL), E. coli DNA Polymerase I (BRL), E. coli RNaseH (BRL) and a supplied buffer in the reaction mixture and incubating at 16° C. for 2 hours, followed by the incubation with T4 DNA Polymerase (BRL). After treating the reaction mixtures with phenol:chloroform (1:1) and precipitating the treated mixtures with ethanol, the precipitates were solubilized in 12 μl of distilled water. A CRNA was synthesized by adding T7 RNA Polymerase (ENZO) and biotinylated UTP and CTP to 2.5 μl of the ds cDNA fraction, and then fragmented by acid treatment.

### Example 3-3

### Hybridization, Washing and Staining on DNA Microarray

Fifteen micrograms of the fragments of these cRNAs were solubilized in a buffer containing 100 mM MES, 1 M [Na+], 20 mM EDTA, and 0.01% Tween 20, and subjected to hybridization on DNA microarrays at 45° C. for 16 hours. In this example, GeneChips: Mu 1KsubA, subB, Mu19KsubA, subB, and subC (Affymetrix Inc.) were used as the microarrays. DNA microarray wash procedure was carried out according to the Affymetrix Fluidics Station protocol. Thereafter, each of the DNA microarrays was stained by antibody fluorescence amplification method as follows. First the DNA microarrays were stained by adding Streptavidin-modified Phycoerythrin (Molecular Probes, Inc.), Then reacted with goat IgG (SIGMA) and biotinylated goat anti-Streptavidin antibody (Vector Laboratories, Ltd.), and re-stained with Streptavidin-modified Phycoerythrin.

### Example 3-4

### DNA Microarray Data Analysis

After the hybridization, washing and staining, the DNA microarrays were subjected to measurement of the fluorescence intensities using GeneChip scanner (Hewlett-Packard). The scanning was repeated two times and the average of the results was stored as image data. Quantification of expression levels and comparative analysis thereof were performed by analyzing the image data by GeneChip software (Affymetrix Inc.). The expression levels of the genes were compared between LY and LY/NR2 and between LY and LY/NR1 by Comparison Analysis of the above software.

As a result, it was an ABCG2 gene of ABC transporter family members that, among approximately 30,000 kinds of genes analyzed concerning their expression levels, showed the most elevated expression selective to LY/NR2 cell line in comparison with that in LY. The ABCG2 gene showed 31-fold increase in expression compared with the parental cell line LY (Table 3). The result suggests that the resistance to Compound-A of each cell line correlated with the increase of this gene expression.

TABLE 3
Analysis of genes with selectively elevated expressions in
mouse Compound-A resistant cell lines by DNA chips

| | Fold increase in resistant | |
|---|---|---|
| | cells^{a} (fold) | |
| Gene Description | LY/NR2 | LY/NR1 |
| ABCG2 | 31.2 | 6.0 |
| af070537 Full Length w/o function | 15.5 | 13.4 |
| ATP Synthetase A chain | 6.2 | NC^{b} |
| Interferon Activatable protein | 5.2 | NC |
| Interferon Activatable protein | 4.7 | 2.3 |
| 3-beta hydroxylstroid dehydrogease | 4.6 | NC |
| Interferon Activatable Protein | 4.5 | 4.7 |
| EST | 4.5 | NC |
| Y13275, Meta-associ tetraspan molecule | 4.3 | 3.8 |
| 19 kD Glycoprotein Autoantigen | 4.2 | 3.2 |
| Lipocortin, x07486 | 4.1 | NC |
| Ig heavy chain precursor | 4.0 | NC |
| Y-box transcription factor | 4.0 | −1.6 |

^{a}The genes that showed more than 4-fold elevated expression in LY/NR2 compared with that in LY cell are listed in decreasing order of their elevated expression. Among these genes, genes displaying the change of expression in LY/NR1 list their value of the elevated expression compared with that in LY cell in the column LY/NRI.
^{b}Genes showing no change of expression compared with that in their parental cells are represented as NC.

### Example 4

### Northern Blot Analysis

To confirm the selective expression of ABCG2 in the resistant cell lines LY/NR1 and Ly/NR2 detected by DNA microarray analysis, Northern blot analysis was performed using cDNA fragment of mouse ABCG2 as a probe, and the same was performed using cDNA fragment of human ABCG2 of the present invention as a probe to detect the expression of human ABCG2 in Compound-A-resistant human cell line.

### Example 4-1

### Preparation of Blots

A total RNA was extracted from each of the cell lines LY, LY/NR1, LY/NR2, HeLaS3, HeLa#7, HCT116, HCT116/NR1, PC-13, and PC-13/NR13 by guanidine isothiocyanate method using ISOGEN reagent (Nippon Gene, Japan). From the obtained total RNA, poly (A)⁺ RNA was purified using FastTrak® 2.0 Kit (Invitrogen Corp.). For detection of each gene's expression, 0.8 μg of the poly (A)⁺ RNA obtained from each cell line was electrophoresed on a formaldehyde denatured agarose gel and transferred to Hybond N+ membrane (Amersham Pharmacia) using 20×SSC buffer for overnight. After transfer, the membranes were air-dried and the nucleic acids on the membranes were UV-fixed by STRATALINKER (Stratagene, Inc.).

### Example 4-2

### Preparation of Probes and Hybridization

For detection of mouse ABCG2, the following procedure was employed to obtain a mouse ABCG2 cDNA fragment used as a probe. PCR amplification of the cDNA was performed using 2 μl of the LY/NR2 cDNA prepared in Example 3 as a template, with an upstream DNA primer (SEQ ID NO:4: CTCATTTAAAAACTTGCTCGGGMCC) and a downstream DNA primer (SEQ ID NO:5: CAAGAGGCCAGMAAGAGCATCATM) synthesized based on the mouse ABCG2 DNA sequence. The 50 μl of PCR reaction mixture was composed of 200 nM each of the synthetic DNA primers, 0.1 mM dNTPs, 0.5 μl of Ex Taq DNA polymerase (TaKaRa, Japan) and 5 μl of a buffer attached to the enzyme. The amplification was performed for 29 cycles using the thermal cycler (PerkinElmer, Inc.). Each cycle consisted of 30 sec at 95° C., 1 min at 60° C., and 2 min at 70° C. Then, after diluting the amplified product, a further PCR amplification was performed using an upstream DNA primer (SEQ ID NO: 6: TACTGGGGCTTATTATTGGTG) and a downstream DNA primer (SEQ ID NO: 7: AAAAGCGATTGTCATGAGAAGTGT) which were annealed to sequences of the amplified product. In the reaction, an amplification was performed for 35 cycles. Each cycle consisted of 30 sec at 95° C., 30 sec at 62° C., and 2 min at 72° C. The amplified products were confirmed by 1% agarose gel electrophoresis and ethidium bromide staining. This mouse ABCG2 cDNA fragment was purified by QIAquick PCR Purification Kit (QIAGEN) and radiolabeled with [α-³²P] dCTP using Multiprime Labelling Kit (Amersham Pharmacia) to prepare a probe for hybridization.

On the other hand, for detection of human ABCG2 gene, PCR amplification was performed using a plasmid having a full length human ABCG2 cDNA of the present invention shown in Example 5, as a template, with an upstream DNA primer (SEQ ID NO:8: CAAAAAGCTTMGACCGAGCTCTATTA AGC) and a downstream DNA primer (SEQ ID NO:9:ATCCTCTAGACCAGG TTTCATGATCCCATTG). The amplified cDNA fragment was radiolabeled by the same method that was performed on the mouse cDNA, to prepare a probe for hybridization. Each of these cDNA probes and 1 mg of salmon sperm DNA were added to the membranes which were prehybridized in QuikHyb hybridization solution (Stratagene) at 65° C. for 30 min, then hybridization was performed at 65° C. for 1 hour. Thereafter, the membranes were washed with 2×SSC containing 0.1% SDS at room temperature for 30 min, then with 0.1×SSC containing 0.1% SDS at room temperature for 15 min, and finally with 0.1×SSC containing 0.1% SDS at 65° C. for 10 min. The radioactivities of the washed membranes were measured by BAS5000 Image Analyzer (Fuji Photo Film Co.) and processed for imaging. In order to clarify that approximately equal amounts of sample RNAs were loaded, each blot was further hybridized with a GAPDH (glyceraldehyde-3-phosphate dehydrogenase) probe: the membranes hybridized with the ABCG2 probe were immersed for 1 min in boiled solutions containing 0.5% SDS and left until they reached room temperature to remove the probes on the membranes. Then the membranes were hybridized with a human GAPDH DNA fragment as a probe, washed and measured by the same conditions that were described in hybridization with ABCG2 probe.

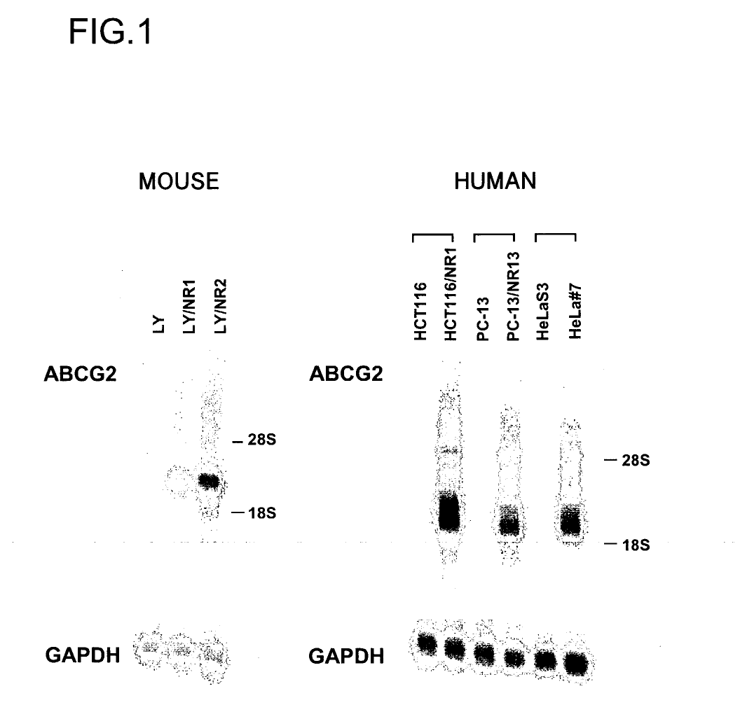

As a result, the mouse cell lines LY, LY/NR1 and LY/NR2 showed an elevated expression of the ABCG2 gene correlated with an increase of resistance, as was demonstrated in the DNA microarray analysis. It was also found that the human ABCG2 gene was selectively overexpressed in all Compound-A-resistant human cell lines such as HCT116/NR1, PC-13/NR13 and HeLa#7, which strongly suggests that the gene of the present invention is involved in Compound-A and Compound-B resistance (FIG. 1).

### Example 5

### Isolation of Full Length Human ABCG2 cDNA from HeLa #7 Cell

### Example 5-1

### Preparation of Synthetic DNA Primer Sequences Based on the Known ABCG2 Sequence

A synthetic DNA primer sequence for PCR, flanking both initiation and termination codons, was prepared based on the sequence reported as ABCP mRNA (GenBank: AF103796).

5′ primer
(SEQ ID NO:8: AAAAAGCTTAAGACCGAGCTCTATTAAGC)
3′ primer
(SEQ ID NO:10: GAATTAAGGGGAAATTTAAGAAT)

In the 5′ primer, eight bases, which are required for digestion by the restriction enzyme Hind III, were added to 5′ end of the sequence derived from ABCP.

### Example 5-2

### Preparation of Total RNA Fraction From HeLa#7 Cell and Synthesis of cDNA

Total RNA was prepared from the cultured human cell line HeLa#7 by guanidine isothiocyanate method using ISOGEN reagent (Nippon Gene, Japan). Then the complementary DNA was synthesized from 2.5 μg of the total RNA fraction by SuperScript II Reverse Transcriptase (BRL) with 18 bases of oligo dT as a primer and a supplied buffer.

### Example 5-3

### Amplification of Human ABCG2 Gene by PCR Using the cDNA Derived from HeLa#7 Cell and Determination of its Nucleotide Sequence

Amplification by PCR using the synthetic primers was performed using 1 μl (125 ng) of the cDNA prepared from the HeLa#7 cells in Example 5-2 as a template. The 50 μl of reaction mixture was composed of 300 nM each of the synthetic DNA primers (SEQ ID NO:8 and No.10), 0.2 mM dNTPs, 2,p1 of LA Taq DNA polymerase (TaKaRa, Japan) and 5 μl of a buffer attached to the enzyme. The amplification was performed 30 cycles using the thermal cycler (PerkinElmer, Inc.). Each cycle consisted of 30 sec at 94° C., 1 min at 55° C., and 4 min at 72° C. The amplified product was confirmed by 1% agarose gel electrophoresis and ethidium bromide staining.

### Example 5-4

### Subcloning of the PCR Product into a Plasmid Vector and Determination of Nucleotide Sequence of the Inserted cDNA Region

The reaction product obtained by PCR in Example 5-3 was separated using 1% agarose gel and the region containing its band was cut out from the gel by a knife, followed by recovery of the DNA using QIAquick Gel Extraction Kit (QIAGEN). After adding A to the recovered DNA at its 3′ end by rTaq DNA polymerase, the DNA was subcloned into a plasmid vector pcDNA3.1N5-His-TOPO according to a method of Eukaryotic TOPO TA cloning Kit (Invitrogen Corp.). E. coli TOP10 competent cells (Invitrogen Corp.) were transformed by the plasmid, then clones having the cDNA insert were selected in LB agar medium containing ampicillin. Emerging colonies were isolated by sterilized picks. Then PCR using a primer set spanning a multicloning site of the plasmid vectors was performed for each clone, and clones having inserts with predicted size were selected. The clones with the PCR product of interest inserted was cultured for overnight in LB medium containing ampicillin, a plasmid DNA was prepared using QIAprep 8 Turbo miniprep kit (QIAGEN). The reaction for determining the nucleotide sequence was performed using DyeDeoxy Terminator Cycle Sequencing Kit (ABI) and the nucleotide sequence was determined by means of a fluorescence automatic sequencer.

As a result, it was found out that the sequence of ABCG2 full length cDNA isolated from HeLa#7 cell was nearly identical to those of ABCG2 cDNAs reported previously except for several essential differences.

The nucleotide sequence of human ABCG2 of the present invention was shown in SEQ ID NO:1 and the E. coli strain, designated as “E. coli HELabcg2”, into which the full length cDNA was cloned was deposited as follows:

(i) Name and Address of the Depositary Institution

NAME: International Patent Organism Depositary, National institute of Advanced Industrial Science and Technology (formerly National Institute of Bioscience and Human-Technology, Agency of Industrial Science and Technology, MITI)

ADDRESS: Tsukuba Central 6,1-1-1 Higashi, Tsukuba, Ibaraki, Japan (postcode: 305-8566)

(ii) Date of Deposit (the original date): Sep. 25, 2000

(iii) Accession number of Domestic Deposit: No.18053 (FERM P-18053)

(iv) Date of Transfer to Budapest Treaty Deposit: Sep. 5, 2001

(v) Accession number of Budapest Treaty Deposit: FERM BP-7726

There are two different ABCG2 genes previously reported about their full length sequences: ABCP and BCRP. These two genes have different parts in their sequences. Where are concerned only the differences of sequences involving in replacement of amino acid, the sequences differ in the regions coding amino acids at positions 24, 166, 208, and 482. ABCP and BCRP code valine and alanine as amino acid at position 24, glutamic acid and glutamine at position 166, serine and phenylalanine at position 208, and arginine and threonine at position 482, respectively.

Among the above four regions, the sequence of the gene the inventors obtained is identical to that of BCRP concerning the regions coding amino acids at positions 24, 166, and 208, i.e. the present invention's gene codes alanine, glutamine, and phenylalanine as amino acids at positions 24, 166, and 208, respectively, while only one region coding amino acid at position 482, i.e. arginine, of the present gene is identical to that of ABCP. Namely, the sequence of the gene the inventors obtained is identical to that reported as BCRP except for one amino acid at position 482, i.e. the gene is a single amino acid substituent of BCRP.

### Example 6

### Amplification of Human ABCG2 cDNA by PCR using a cDNA Derived from Normal Human Tissue and Determination of the Nucleotide Sequence Thereof

Concerning region different from the sequences of ABCG2 or BCRP, the nuclleotide sequence of ABCG2 cDNA derived from normal human tissue was determined using direct sequencing analysis.

PCR amplification was performed using synthetic DNA primers (SEQ ID NO:8: CAAAAAGCTTMGACCGAGCTCTATTMGC and SEQ ID NO:11: AGAGATCGATGCCCTGCTTTACCA), and 2.5 μl (˜0.5 ng) of cDNA derived from placenta and kidney contained in Human MTCTM Panel I (Clontec) as a template. The 50 μl of PCR reaction mixture was composed of 200 nM each of the synthetic DNA primers (SEQ ID NO:8 and No.11), 0.4 mM dNTPs, 0.5 μl of LA Taq DNA polymerase and 5 μl of a buffer attached to the enzyme. The amplification reaction was performed for 35 cycles using the thermal cycler (PerkinElmer, Inc.). Each cycle consisted of 30 sec at 94° C., 1 min at 55° C., and 3 min at 72° C. The amplified product was confirmed by 1% agarose gel electrophoresis and ethidium bromide staining. The PCR product was purified by QIAquick PCR Purification Kit (QIAGEN), then subjected to direct sequencing. The reaction for determining the nucleotide sequence, as performed in Example 5-4, was performed using DyeDeoxy Terminator Cycle Sequencing Kit (ABI) and the sequence was determined by means of a fluorescence automatic sequencer.

As a result, it was found out that in the ABCG2 cDNA derived from human placenta and kidney, the regions coding amino acids at positions 24, 166, 208, and 482 code alanine, glutamine, phenylalanine, and arginine, respectively, which suggests that the same sequence as the inventors obtained from HeLa#7 cells is also expressed in normal human tissue.

### Example 7

### Preparation of Human ABCG2 Expressing Cells

As shown in Example 5, the human ABCG2 full length cDNA from HeLa#7 cell was inserted into a plasmid for expression in mammalian cells: pcDNA3.1N5-His-TOPO (Invitrogen Corp.). PC-13 cells were transfected with the ABCG2 expression plasmids using Effectene Transfection Reagent (QIAGEN). The experiment was performed according to the attached procedural manual. As a control experiment, other PC-13 cells were transfected with vector alone without the ABCG2 sequence. The two experiments were conducted simultaneously. Two days after the transfection, the original media were replaced with selection media containing 0.2 mg/ml of Geneticin (GIBCO BRL). By culture for a long time under this condition, stable transfectant clones with the introduced plasmids were selected. Two weeks from the transfection, emerging colonies were picked and expression of ABCG2 in the cells was examined by Northern Blot analysis. Several clones with high level of expression of ABCG2 such as clones PC-13/ABCG2-2, PC13/ABCG2-3 were selected and subjected to subsequent analysis. Northern Blot analysis was performed as described in Example 4:8 μg of total RNA prepared from each clone using RNeasy mini kit (QIAGEN) was separated on a formaldehyde denatured agarose gel, followed by analysis using human ABCG2 cDNA fragment of the present invention as a probe.

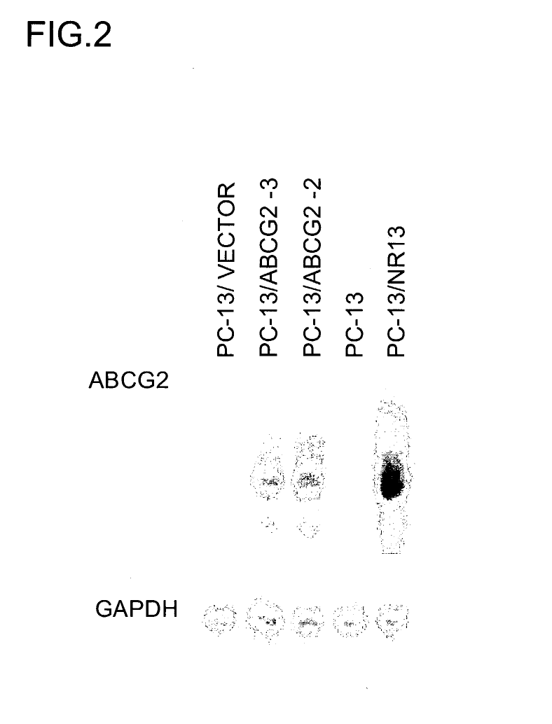

As a result, it was found that the clones PC-13/ABCG2-2 and PC-13/ABCG2-3 highly expressed a transcript of the present invention's ABCG2. The expression level in the clone PC-13/ABCG2-2 was about 35% of that in the resistant cell line PC-13/NR13 and the expression level in the clone PC-13/ABCG2-3 was about 20% of that in the resistant cell line PC-13/NR13 (FIG. 2).

### Example 8

### Measurement of Drug Sensitivity to Various Cancer

Chemotherapeutic Drugs of Human ABCG2 Expressing Cells

The drug sensitivity of the stable transfectant clone, PC-13 cell with transfected human ABCG2 expressing plasmid, was measured by SRB method described in Example 2.

As a result, the PC-13 cell with introduced vector alone did not show resistance to the two compounds (Compound-A and Compound-B). On the other hand, the clone PC-13/ABCG2-2 with the highest expression of introduced ABCG2 showed 22- and 17-fold resistance to Compound-A and Compound-B, respectively, and the clone PC-13/ABCG2-3 with the second highest expression showed 9- and 11.7-fold resistance to Compound-A and Compound-B, respectively, compared to the PC-13 cell with introduced vector alone. These results strongly suggest that the overexpression of ABCG2 confers resistance to these indolocarbazole compounds on the cells. However, these PC-13 cell lines with introduced ABCG2 did not display remarkable resistance to the other agents, i.e. camptothecin, topotecan, mitoxantrone, and etoposide (table 4). PC-13/ABCG2-2 and PC-13/ABCG2-3 displayed 0.42- and 0.60-fold resistance, respectively, to mitoxantrone compared to the PC-13 with introduced vector alone, which is much different from the report that MCF-7 cells expressing introduced BCRP showed 30-fold resistance to mitoxantrone (Doyle, A. et al., Proc. Natl. Acad. Sci. U.S.A. 95, 15665-15670 (1998)).

TABLE 4
Drug sensitivities of PC-13 cell lines transfected with ABCG2 expression vectors

| | PC-13/Vector | PC-13/ABCG2-2 | | PC-13/ABCG2-3 | |
|---|---|---|---|---|---|
| Drug | IC50 (μM) | IC50 (μM) | Rel. Res. | IC50 (μM) | Rel. Res. |
| Compound-A | 0.30 ± 0.12 | 6.60 ± 2.6 | 22.0 | 2.7 ± 1.0 | 9.0 |
| Compound-B | 0.012 ± 0.004 | 0.20 ± 0.03 | 17 | 0.14 ± 0.01 | 11.7 |
| Camptothecin | 0.043 ± 0.011 | 0.029 ± 0.005 | 0.67 | 0.052 ± 0.008 | 1.2 |
| Mitoxantrone | 0.052 ± 0.021 | 0.022 ± 0.008 | 0.42 | 0.031 ± 0.018 | 0.60 |
| Topotecan | 0.21 ± 0.040 | 0.19 ± 0.05 | 0.90 | 0.096 ± 0.022 | 0.46 |
| Etoposide | 1.0 ± 0.29 | 0.46 ± 0.075 | 0.46 | 1.1 ± 0.33 | 1.1 |

Each of the data was shown as average value ± a standard error of three independent experiments. The relative resistances of PC-13/ABCG2-2 and PC-13/ABCG2-3 were obtained by dividing IC50 value of the resistant cells by IC50 value of the vector cells.

### Example 9

### Analysis of Indolocarbazole Compound Accumulation in Cells Expressing Human ABCG2 of the Present Invention

The amount of accumulation of Compound-B in stable transfectant clone, PC-13 cell with transfected ABCG2 expression plasmid were measured as follows.

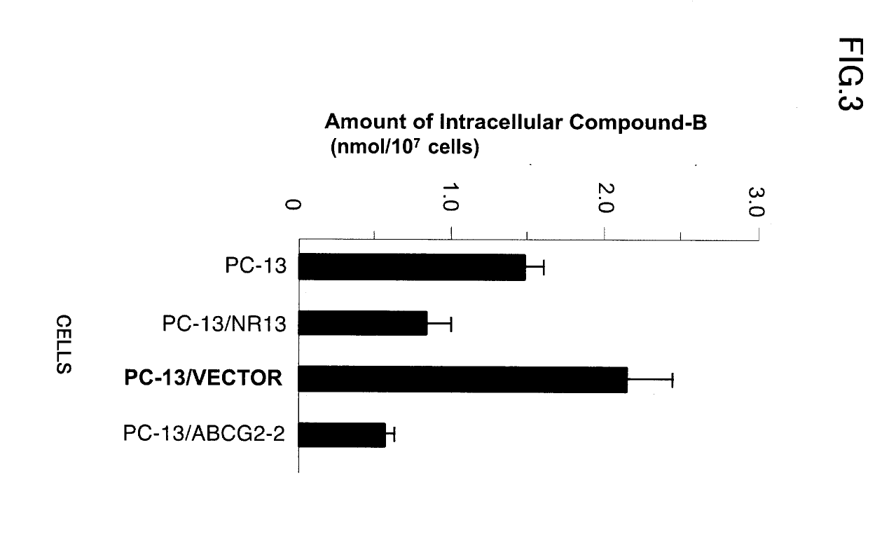

The cells seeded at a density of 1.5×10⁶ in a 25-cm² culture flask were incubated for overnight. On the following day, the original medium was replaced with a medium containing 50 μM [¹⁴C]-labeled Compound-B, followed by culture at 37° C. for 120 min in a CO₂ incubator to incorporate the labeled compound into the cells. After incubation for 120 min, the cells were rapidly chilled on ice, washed with PBS, detached using 2.5% trypsin. The lysate was centrifuged at 400×g at 4° C. for 3 min to collect the cells. The cells again suspended in PBS were counted and centrifuged under the same conditions that were described above to recover the cells again. The cells were lysed by Triton X-100 and centrifuged at 2000×g at 4° C. for 15 min to collect the fractions of the membrane and cytoplasm as supernatant. The remaining pellet Was solubilized in 0.2 N NaOH to lyse the nuclei. The obtained supernatant and nuclei samples were supplemented with Clear-sol I (Nacalai Tesque, Inc.) and separately counted in a liquid scintillation counter: TRICARB2300 (Packerd). The values of the supernatant and nuclei samples were added together as Intracellular Accumulation (FIG. 3).

As a result of this analysis, the accumulation of Compound-B in PC-13/ABCG2-2 cells with introduced the ABCG2 expression plasmid is almost the same level as that in the Compound-A resistant cell PC-13/NR13 cell line, while the value was about only one quarter of that in cells with introduced vector alone. The result shows that the cells expressing ABCG2 with the sequence identified by the present inventors displayed an extremely reduced accumulation of Compound-B, which strongly suggests the efflux of indolocarbazole compounds by genes of the present application.

### Example 10

### Preparation of ABCG2-482T Sequence by Introducing a base Substitution

In order to clearly differentiate between the activities of the present invention's ABCG2 (SEQ ID NO:1) whose codon at position 482 codes arginine and the gene reported above as BCRP, reported by Doyle et al., whose codon at position 482 codes threonine, a BCRP-type full length ABCG2 cDNA with a single base substitution introduced was prepared, according to the following procedure, from the cDNA sequence of the cloned ABCG2 gene in Example 5, designated as ABCG2-482T (SEQ ID NO: 12). Note that in reference to amino acid sequence, the sequence of ABCG2-482T is the same as that of the gene registered as BCRP, although in reference to nucleotide sequence, there is another position a single base is changed without substitution of amino acid.

### Example 10-1

### Preparation of a Synthesized cDNA Primer Sequence with a Single Base Substitution Introduced in Order to Change to ABCG2-482T Sequence

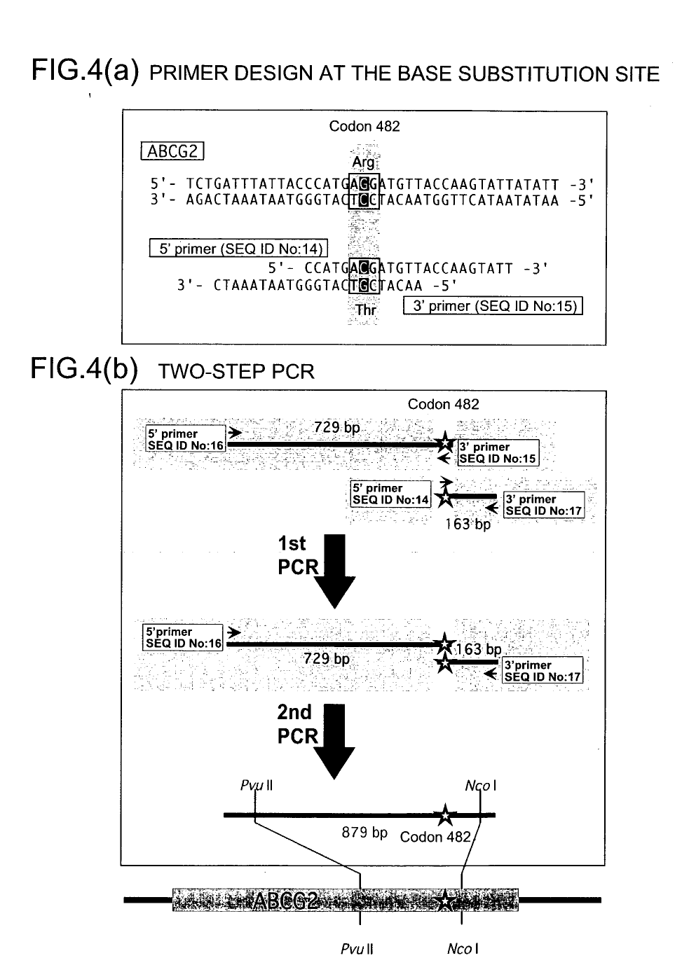

Firstly, based on the cDNA sequence of the present invention's ABCG2 gene, two DNA primers for PCR were synthesized. Each of the two primers had a sequence in which the nucleotide sequence of the ABCG2 gene's codon at position 482 was replaced with the corresponding region of the nucleotide sequence reported as BCRP, and could anneal the corresponding strand of the ABCG2 cDNA (see FIG. 4-(a)). Note that the lowercase character in the following sequence of each primer represents a single base substitution.

| | |
|---|---|
| 5′ primer | |
| (SEQ ID NO: 14: | CCATGAcGATGTTACCAAGTATT) |
| 3′ primer | |
| (SEQ ID NO: 15: | AACATCgTCATGGGTAATAAATC) |

### Example 10-2

### Introduction of a Single Base Substitution by Two Steps PCR

Next, cDNA fragments of the upstream region and of the downstream region, each of which contained the region where the substitution should be introduced, were separately prepared by PCR using the plasmid encoding the ABCG2 gene cDNA in Example 5 as a template.

The cDNA fragment of the upstream region was amplified by PCR using a 5′ primer (SEQ ID NO: 16: CATTCATCAGCCTCGATATTCCA) and a 3′ primer (SEQ ID NO: 15: AACATCgTCATGGGTMTAAATC) prepared in Example 10-1. The cDNA fragment of the downstream region was amplified by PCR using a 5′ primer prepared in Example 10-1 (SEQ ID NO: 14: CCATGAcGATGTTACCMGTATT) and a 3′ primer (SEQ ID NO: 17: ACCACACTCTGACCTGCTGCTA) (FIG. 4-(b), 1st PCR).

So as to link between these two cDNA fragments, the mixture of the two cDNAs was then amplified by PCR using a 5′ primer (SEQ ID NO: 16: CATTCATCAGCCTCGATATTCCA) and a 3′ primer (SEQ ID NO: 17: ACCACACTCTGACCTGCTGCTA) (FIG. 4-(b), 2nd PCR) so that a PCR product including the base substitute region inside was obtained. After PCR, the reaction product was separated using 1% agarose gel and the region containing its band was cut out from the gel by a knife, followed by collection of the DNA using QIAquick Gel Extraction Kit (QIAGEN). After adding A to the collected DNA at its 3′ end by rtaq DNA polymerase, the DNA was subcloned into a plasmid vector pCR2.1-TOPO according to a protocol of Eukaryotic TOPO TA cloning Kit (Invitrogen Corp.). E. coli TOP10 competent cells (Invitrogen Corp.) were transformed by the plasmids, then clones having the cDNA insert were selected in LB agar medium containing ampicillin. Emerging colonies were isolated by sterilized picks. PCR using a primer set spanning a multicloning site of a plasmid vectors was then performed for each clone, and clones having an inserts with predicted size were selected. The clones with the PCR product of interest inserted was cultured for overnight in LB medium containing ampicillin, a plasmid DNA was prepared using QIAprep 8 Turbo miniprep kit (QIAGEN). A reaction for determining the nucleotide sequence was performed using DyeDeoxy Terminator Cycle Sequencing Kit (ABI) and the nucleotide sequence was decoded by means of a fluorescence automatic sequencer. As a result, the introduction of a single base substitution was revealed.

### Example 10-3

### Preparation of Full Length ABCG2-482T cDNA and Introduction thereof into a Plasmid Capable of Expressing in Animal Cells

The cDNA fragment with a single base substitution introduced prepared in Example 10-2 was digested by two restriction enzymes, Pvu II and Nco I, which cut the inside of the fragment, and the fragment including the region with the introduced substitution was isolated. By ligating this fragment to a fragment obtained by digesting the ABCG2 cDNA cloned in Example 5 using Pvu II and Nco I, a full length ABCG2 cDNA was obtained whose region including codon 482 was replaced with a fragment in which the substitution was introduced. The full length cDNA was designated as ABCG2-482T.

By inserting the ABCG2-482T cDNA into a plasmid vector pcDNA3.1/Myc-His, a plasmid expressing in animal cells was prepared. E. coli TOP10 competent cells (Invitrogen Corp.) were transformed by the plasmids, then clones having the cDNA insert were selected in LB agar medium containing ampicillin. Emerging colonies were isolated by sterilized picks. PCR using a primer set spanning a multicloning site of the plasmid vectors was then performed for each clone, and clones having insert with predicted size were selected. The clones with the PCR product of interest inserted was cultured for overnight in LB medium containing ampicillin, a plasmid DNA was prepared using QIAprep 8 Turbo miniprep kit (QIAGEN). A reaction for determining the nucleotide sequence was performed using DyeDeoxy Terminator Cycle Sequencing Kit (ABI) and the nucleotide sequence was decoded by means of a fluorescence automatic sequencer, confirming that the plasmid had the sequence of ABCG2-482T.

### Example 11

### Preparation of MCF-7 Cell which Expresses ABCG2 of the Present Invention or ABCG2-482T

The full length human ABCG2 cDNA of the present invention prepared in Example 5 was inserted into a plasmid for expression in mammalian cells, followed by transfection of human breast cancer cells, MCF-7 cells, with the ABCG2 expression plasmid using Effectene Transfection Reagent (QIAGEN). Likewise, human breast cancer cells, MCF-7 cells, were transfected with a plasmid including the ABCG2-482T cDNA prepared in Example 10. The experiments were carried out according to an attached procedural manual. As a control experiment, the MCF-7 cells were also transfected with vector alone. The three experiments were conducted simultaneously. Two days from the transfection, the original media were replaced with selective media containing 900 μg/ml of Geneticin (GIBCO BRL). By culture over a prolonged term under this condition, stable transfectant clones with introduced plasmid were selected. Two to three weeks from the transfection, emerging colonies were picked and the expression of ABCG2 in the cells was examined by Northern Blot analysis. Northern Blot analysis was performed as described in Example 4: 8 μg of total RNA prepared from each clone using RNeasy mini kit (QIAGEN) was separated on a formaldehyde denatured agarose gel, followed by analysis using the human ABCG2 cDNA fragment of the present invention as a probe.

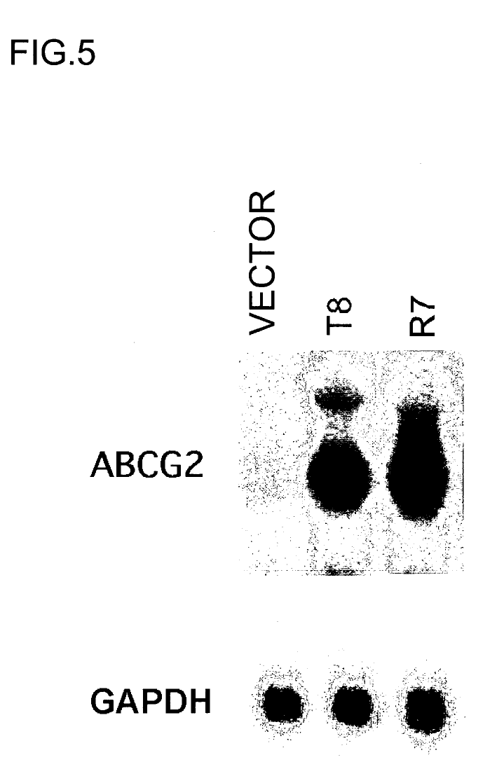

As a result, it was found that MCF-7/R7 among the clones transfected with ABCG2 of the present invention and MCF-7/T8 among the clones transfected with ABCG2-482T, a single amino acid substituent, showed high expression of their respective introduced genes. The two clones were subjected to subsequent analysis. The two clones displayed almost the same amounts of expression of ABCG2 (FIG. 5).

### Example 12

### Measurement of Drug Sensitivity to Various Cancer Chemotherapeutic Drugs of MCF-7 Cell which Expresses ABCG2 of the Present Invention or ABCG2-482T

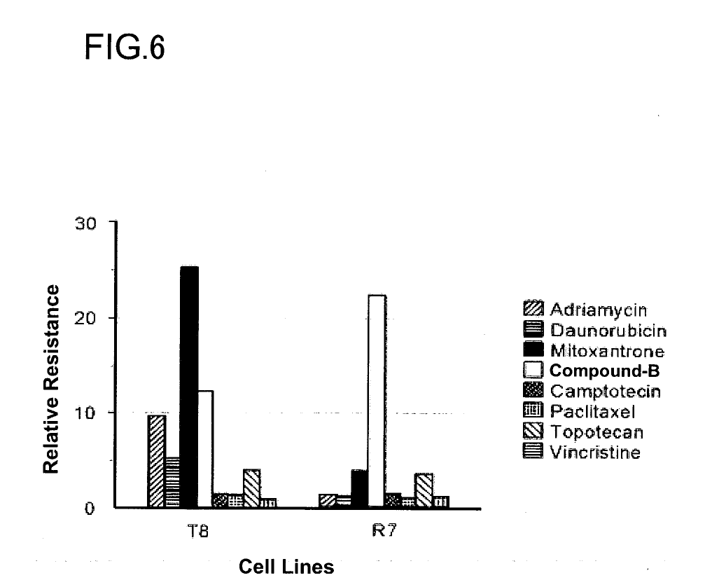

The drug sensitivity of the stable transfectant clones which were established from MCF-7 cells with introduced ABCG2- or ABCG2-482T-expression plasmid, was measured using SRB method as described in Example 2 and the sensitivity of each transfectant to drugs was shown as a relative resistance to the cells with introduced vector alone (FIG. 6).

As a result, MCF-7/T8 with introduced ABCG2-482T displayed 12-fold resistance to Compound-B, and also showed resistance to several compounds, i.e., 25-, 9.6-, and 5.1-fold resistance to Mitoxantrone, Adriamycin, and Daunorubicin, respectively. Namely, as was reported previously as BCRP, it was demonstrated that the cell expressing genes whose codon 482 codes threonine has a property as a multidrug resistance factor, i.e. responsible for resistance to various compounds.

On the contrary, MCF-7/R7 with introduced ABCG2 of the present invention showed high resistance, i.e. 22-fold resistance to Compound-B, but displayed mere 3.8-fold resistance to Mitoxantrone and no resistance to Adriamycin and Daunorubicin. Namely, it was found out that cells expressing the present invention's ABCG2 whose codon 482 codes arginine is selectively resistant to Compound-B.

As was described above, it was demonstrated that ABCG2 of the type reported as BCRP confers resistance to a broad range of chemotherapeutic drugs, while the present invention's ABCG2 different from BCRP-ABCG2 in one amino acid confers resistance selective to indolocarbazole.

### Example 13

### Analysis of Indolocarbazole Compound Accumulation in MCF-7 Cell which Expresses ABCG2 of the Present Invention or ABCG2-482T

The amount of accumulation of Compound-B in the MCF-7 stable transfectant clones with introduced ABCG2 of the present invention or ABCG2-482T was measured as follows.

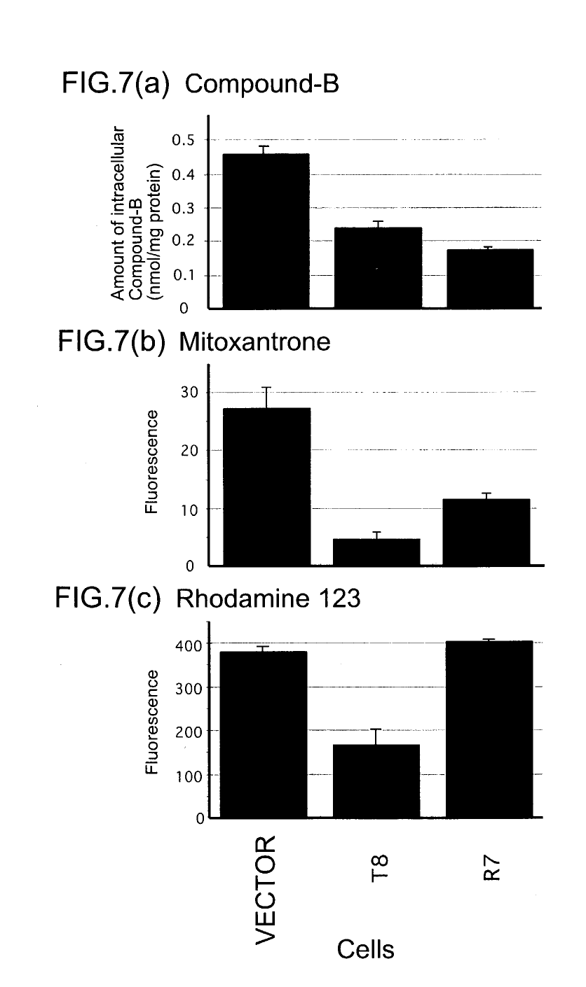

Cells seeded at 8×10⁵ cells/well in a 6-well culture plate were incubated for overnight. The next day, the cells in a fresh medium containing 7 μM [¹⁴C]-labeled Compound-B were cultured at 37° C. for 180 min in a CO₂ incubator to incorporate the labeled compound into the cells. After 180 min incubation, the cells were rapidly chilled on ice, washed with PBS three times, then the cells were lysed by adding 2N NaOH and incubating at 40° C. for 60 min with shaking. Portions of the obtained lysate samples were subjected to the determination of protein concentration with Bradford method. The remaining parts of the samples were measured in a liquid scintillation counter: TRICARB2500 (Packerd) after addition of Hionic-fluor (Packerd). The result was indicated as Intracellular Accumulation (FIG. 7-(a)).

As a result of this analysis, MCF-7/T8 with introduced ABCG2-482T and MCF-7/R7 with introduced the present invention's ABCG2 both showed reduced accumulation of Compound-B, which proved that the cell with introduced ABCG2-482T and that with introduced the present invention's gene both perform active efflux of indolocarbazole compounds.

### Example 14

### Analysis of Mitoxantrone and Rhodamine Accumulation in MCF-7 Cell which Expresses ABCG2 of the Present Invention or ABCG2-482T

The amount of accumulation of Mitoxantrone in the MCF-7 stable transfectants with introduced ABCG2 of the present invention or ABCG2-482T was measured as follows.

Cells seeded at 1.0×10⁶ cells/well in a 6-well culture plate were incubated for overnight. The next day, the cells in a fresh medium containing 20 μM Mitoxantrone were cultured at 37° C. for 90 min in a CO₂ incubator to incorporate the compound into the cells. After a lapse of 90 min, the cells were rapidly chilled on ice, washed once with PBS, detached using 2.5% trypsin. The lysate was centrifuged at 400×g at 4° C. for 3 min to collect the cells. The collected cells were suspended in Hank's Balanced Salt Solutions containing 1% BSA and subjected to fluorimetry of the compound using a flow cytometer, Epics Elite (Beckman Coulter, Inc.). The value was indicated as Intracellular Accumulation (FIG. 7-(b)).

On the other hand, the amount of intracellular accumulation of Rhodamine in the MCF-7 stable transfectant clones with introduced ABCG2 of the present invention or ABCG2-482T was measured as follows.

Cells seeded at 1.0×10⁶ cells/well in a 6-well culture plate were incubated for overnight. The next day, the cells in a fresh medium containing 5 μM Rhodamine were cultured at 37° C. for 30 min in a CO₂ incubator to incorporate the compound into the cells. After a lapse of 30 min, the cells were rapidly chilled on ice, washed once with PBS, detached using 2.5% trypsin. The lysate was centrifuged at 400×g at 4° C. for 3 min to collect the cells. The collected cells were suspended in Hank's Balanced Salt Solutions with 1% BSA and subjected to fluorometry of the compound using a flow cytometer, FACSCalibur (Becton Dickinson and Company). The value was indicated as Intracellular Accumulation (FIG. 7-(c)).

As a result of this analysis, it was found out that accumulation of Mitoxantrone in MCF-7/T8 cell was reduced to about 20% of that in the cell with introduced vector alone, suggesting strong efflux of Mitoxantrone in MCF-7/T8 cell, whereas the accumulation in MCF-7/R7 cell was only reduced to about 50% of that in the cell with introduced vector alone, suggesting not so strong efflux in MCF-7/R7 cell. On the contrary, it was found out that accumulation of Rhodamine in MCF-7/T8 cell was reduced to about 50% of that in the cell with introduced vector alone, whereas the accumulation in MCF-7/R7 cell was almost the same level as that in the cell with introduced vector alone, suggesting no efflux of Rhodamine in MCF-7/R7 cell.

As was described above, it was demonstrated that attributed by substitution of a single amino acid, ABCG2 of the present invention and ABCG2-482T are different in their substrate-selectivity from each other.

### INDUSTRIAL APPLICABILITY

The present invention provides a protein that selectively transports indolocarbazole compounds outside the cell and the gene thereof. This enables screening of inhibitors using the transporter protein and the gene coding it and diagnosis on the suitability for administration of an anticancer drug. In addition, its application to treatment for cancer can be expected because inhibitors to be obtained can enhance the sensitivity of cancer cells to anticancer drugs.

1. An isolated polypeptide comprising (a) the amino acid sequence of SEQ ID NO:2; (b) at least 15 contiguous amino acids of SEQ ID NO:2 and the amino acid corresponding to position 482 in SEQ ID NO:2; or (c) an amino acid sequence that is at least 70% identical to SEQ ID NO:2, which confers on a mammalian cell, a selective resistance to a compound of formula (I): 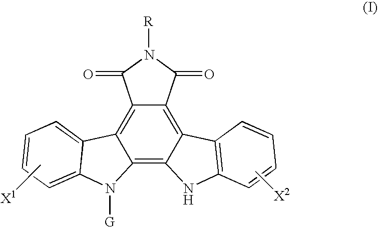
wherein X¹ is a 2-hydroxyl group, X² is a 10-hydroxyl group; R is (1-hydroxymethyl-2-hydroxyl) ethylamino group, and G is a β-D-glucopyranosyl group.

2. An isolated polynucleotide, which encodes the isolated polypeptide of claim 1, or a complementary polynucleotide thereto.

3. The isolated polynucleotide of claim 2, which comprises SEQ ID NO:1 or at least 45 continuous nucleotides of SEQ ID NO:1, or the complementary polynucleotide thereto.

4. A recombinant vector comprising the isolated polynucleotide of claim 2.

5. A recombinant vector comprising the isolated polynucleotide of claim 3.

6. A transformed cell comprising the vector of claim 4.

7. A transformed cell comprising the vector of claim 5.

8. A method of producing an ABCG2 transporter protein or a portion thereof, comprising culturing the transformed cell of claim 6 or 7, and recovering the protein produced.

9. An antibody, which binds to the isolated polypeptide of claim 1.

10. A method of measuring the resistance of a mammalian cell to a cancer chemotherapeutic drug, comprising measuring the expression of the isolated polypeptide of claim 1 in the mammalian cell, wherein an increased level of the protein expression relative to a control mammalian cell that is not resistant to the cancer chemotherapeutic drug is indicative that the mammalian cell is resistant to the chemotherapeutic drug.

11. The method of claim 10, wherein the chemotherapeutic drug is represented by formula (I): 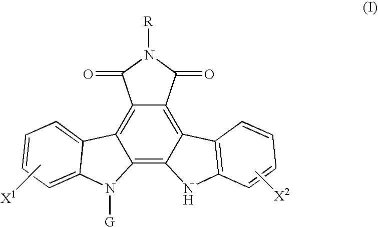
wherein X¹ and X² each independently represent a hydrogen atom, a halogen atom or a hydroxyl group;
R represents a hydrogen atom, an amino, a formylamino, or a lower alkylamino, which may be substituted with any one selected from the group consisting of one to three hydroxyl groups, a pyridyl group optionally having substituents, and thienyl group optionally having substituents; and
G represents a pentose group, a hexose group or a derivative thereof which may be substituted with an amino group.

12. The method of claim 11, wherein
X¹ and X² each independently represent a halogen atom or a hydroxyl group;
R represents a hydrogen atom, a formylamino, or a lower alkylamino which may be substituted with any one selected from the group consisting of one to three hydroxyl groups, a pyridyl group optionally having substituents, and thienyl group optionally having substituents; and
G represents a hexose group which may be substituted with an amino group.

13. The method of claim 11, wherein X¹ represents 2-hydroxyl group, X² represents 10-hydroxyl group, R represents (1-hydroxymethyl-2-hydroxyl) ethylamino group, and G represents β-D-glucopyranosyl.

14. The method of claim 10, wherein the expression of the polypeptide is measured with an antibody that specifically binds to the polypeptide.

15. The method of claim 10, wherein the expression of the polypeptide is measured with an isolated polynucleotide which encodes a polypeptide comprising (a) the amino acid sequence of SEQ ID NO:2; (b)at least 15 contiguous amino acids of SEQ ID NO:2 and the amino acid corresponding to position 482 in SEQ ID NO:2; or (c) an amino acid sequence that is at least 70% identical to SEQ ID NO:2, which confers on a mammalian cell, a selective resistance to a compound of formula (I): 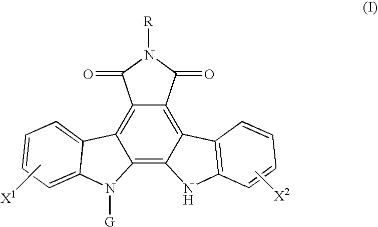
wherein X¹ is a 2-hydroxyl group, X² is a 10-hydroxyl group; R is (1-hydroxymethyl-2-hydroxyl) ethylamino group, and G is a β-D-glucopyranosyl group.

16. A method of predicting the resistance of a patient to a cancer chemotherapeutic drug, comprising measuring the expression of the isolated polypeptide of claim 1 in the mammalian cell, wherein an increased level of the protein expression relative to a control sample that is not resistant to the cancer chemotherapeutic drug is indicative of resistance.

17. The method of claim 16, wherein the chemotherapeutic drug is represented by formula (I): 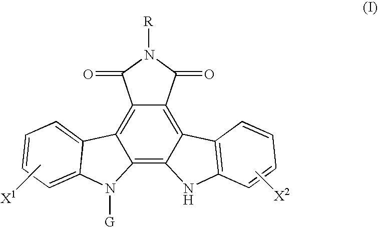
wherein X¹ and X² each independently represent a hydrogen atom, a halogen atom or a hydroxyl group;
R represents a hydrogen atom, an amino, a formylamino, or a lower alkylamino, which may be substituted with any one selected from the group consisting of one to three hydroxyl groups, a pyridyl group optionally having substituents, and thienyl group optionally having substituents; and
G represents a pentose group, a hexose group or a derivative thereof which may be substituted with an amino group.

18. The method of claim 17, wherein
X¹ and X² each independently represent a halogen atom or a hydroxyl group;
R represents a hydrogen atom, a formylamino, or a lower alkylamino which may be substituted with any one selected from the group consisting of one to three hydroxyl groups, a pyridyl group optionally having substituents, and thienyl group optionally having substituents; and
G represents a hexose group which may be substituted with an amino group.

19. The method of claim 17, wherein X¹ represents 2-hydroxyl group, X² represents 10-hydroxyl group, R represents (1-hydroxymethyl-2-hydroxyl) ethylamino group, and G represents β-D-glucopyranosyl.

20. A method of screening for a substance that inhibits the function of the polypeptide of claim 1, comprising
contacting the polypeptide with a compound of formula (I) and a substance to be screened; and
measuring the activity of the protein, wherein a decrease in the binding between the polypeptide and the compound of formula (I), a decrease of the ATP degrading activity when the compound of formula (I) binds thereto, and/or a decrease in the transporting the compound of formula (I) indicates that the substance inhibits the function of the polypeptide: 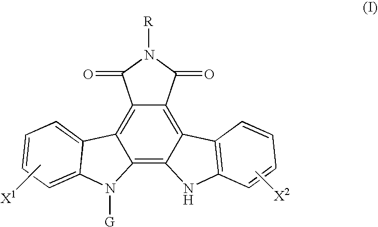
wherein X¹ and X² each independently represent a hydrogen atom, a halogen atom or a hydroxyl group;
R represents a hydrogen atom, an amino, a formylamino, or a lower alkylamino, which may be substituted with any one selected from the group consisting of one to three hydroxyl groups, a pyridyl group optionally having substituents, and thienyl group optionally having substituents; and
G represents a pentose group, a hexose group or a derivative thereof which may be substituted with an amino group.

21. The method of claim 20, wherein
X¹ and X² each independently represent a halogen atom or a hydroxyl group;
R represents a hydrogen atom, a formylamino, or a lower alkylamino which may be substituted with any one selected from the group consisting of one to three hydroxyl groups, a pyridyl group optionally having substituents, and thienyl group optionally having substituents; and
G represents a hexose group which may be substituted with an amino group.

22. The method of claim 20, wherein X¹ represents 2-hydroxyl group, X² represents 10-hydroxyl group, R represents (1-hydroxymethyl-2-hydroxyl) ethylamino group, and G represents β-D-glucopyranosyl.

23. A substance identified by the method of claim 20.

24. A composition comprising the substance of claim 23 and a carrier.

25. In a method of treating a patient with a compound of formula (I), the improvement comprising
administering a substance of claim 23 and the compound of formula (I) to the patient: 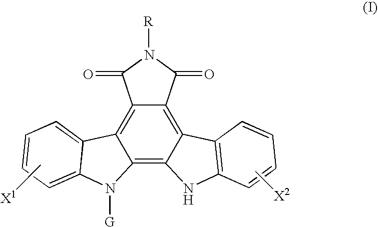
wherein X¹ and X² each independently represent a hydrogen atom, a halogen atom or a hydroxyl group;
R represents a hydrogen atom, an amino, a formylamino, or a lower alkylamino, which may be substituted with any one selected from the group consisting of one to three hydroxyl groups, a pyridyl group optionally having substituents, and thienyl group optionally having substituents; and
G represents a pentose group, a hexose group or a derivative thereof which may be substituted with an amino group.

26. The method of claim 25, wherein
X¹ and X² each independently represent a halogen atom or a hydroxyl group;
R represents a hydrogen atom, a formylamino, or a lower alkylamino which may be substituted with any one selected from the group consisting of one to three hydroxyl groups, a pyridyl group optionally having substituents, and thienyl group optionally having substituents; and
G represents a hexose group which may be substituted with an amino group.

27. The method of claim 25, wherein X¹ represents 2-hydroxyl group, X² represents 10-hydroxyl group, R represents (1-hydroxymethyl-2-hydroxyl) ethylamino group, and G represents β-D-glucopyranosyl.

28. A method of screening a substance which inhibits the function of the polypeptide claim 1, comprising
contacting a mammalian cell that expresses the polypeptide with a compound of formula (I) and a substance to be screened; and
measuring the activity of the protein, wherein an enhancement of the toxicity of the compound of formula (I) against the mammalian cell indicates that the substance inhibits the function of the polypeptide: 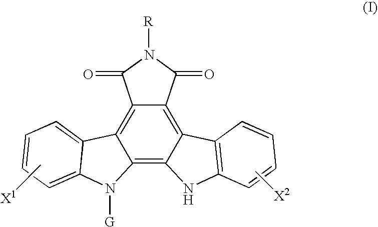
wherein X¹ and X² each independently represent a hydrogen atom, a halogen atom or a hydroxyl group;
R represents a hydrogen atom, an amino, a formylamino, or a lower alkylamino, which may be substituted with any one selected from the group consisting of one to three hydroxyl groups, a pyridyl group optionally having substituents, and thienyl group optionally having substituents; and
G represents a pentose group, a hexose group or a derivative thereof which may be substituted with an amino group.

29. The method of claim 28, wherein
X¹ and X² each independently represent a halogen atom or a hydroxyl group;
R represents a hydrogen atom, a formylamino, or a lower alkylamino which may be substituted with any one selected from the group consisting of one to three hydroxyl groups, a pyridyl group optionally having substituents, and thienyl group optionally having substituents; and
G represents a hexose group which may be substituted with an amino group.

30. The method of claim 28, wherein X¹ represents 2-hydroxyl group, X² represents 10-hydroxyl group, R represents (1-hydroxymethyl-2-hydroxyl) ethylamino group, and G represents β-D-glucopyranosyl.

31. A substance identified by the method of claim 28.

32. A composition comprising the substance of claim 31 and a carrier.

33. In a method of treating a patient with a compound of formula (I), the improvement comprising
administering a substance of claim 31 and the compound of formula (I) to the patient: 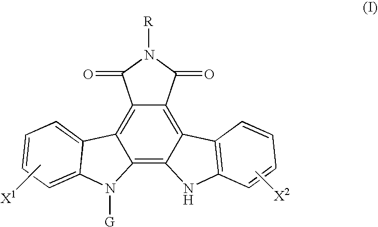
wherein X¹ and X² each independently represent a hydrogen atom, a halogen atom or a hydroxyl group;
R represents a hydrogen atom, an amino, a formylamino, or a lower alkylamino, which may be substituted with any one selected from the group consisting of one to three hydroxyl groups, a pyridyl group optionally having substituents, and thienyl group optionally having substituents; and
G represents a pentose group, a hexose group or a derivative thereof which may be substituted with an amino group.

34. The method of claim 33, wherein
X¹ and X² each independently represent a halogen atom or a hydroxyl group;
R represents a hydrogen atom, a formylamino, or a lower alkylamino which may be substituted with any one selected from the group consisting of one to three hydroxyl groups, a pyridyl group optionally having substituents, and thienyl group optionally having substituents; and
G represents a hexose group which may be substituted with an amino group.

35. The method of claim 33, wherein X¹ represents 2-hydroxyl group, X² represents 10-hydroxyl group, R represents (1-hydroxymethyl-2-hydroxyl) ethylamino group, and G represents β-D-glucopyranosyl.

36. An antisense nucleotide sequence, which inhibits the expression of the polynucleotide of claim 2.

37. A method of inhibiting the expression of the polynucleotide of claim 2 in a mammalian cell, comprising administering or expressing an antisense nucleotide sequence, which inhibits the expression of the polynucleotide to the mammalian cell.

38. In a method of treating a patient with a compound of formula (I), the improvement comprising
inhibiting the expression of the polynucleotide of claim 2 by administering or expressing an antisense nucleotide sequence, which inhibits the expression of the polynucleotide: 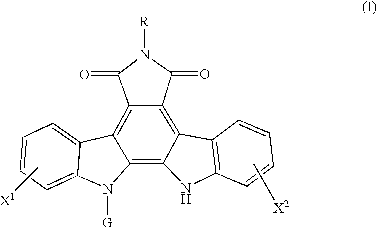
wherein X¹ and X² each independently represent a hydrogen atom, a halogen atom or a hydroxyl group;
R represents a hydrogen atom, an amino, a formylamino, or a lower alkylamino, which may be substituted with any one selected from the group consisting of one to three hydroxyl groups, a pyridyl group optionally having substituents, and thienyl group optionally having substituents; and
G represents a pentose group, a hexose group or a derivative thereof which may be substituted with an amino group.

39. The method of claim 38, wherein
X¹ and X² each independently represent a halogen atom or a hydroxyl group;
R represents a hydrogen atom, a formylamino, or a lower alkylamino which may be substituted with any one selected from the group consisting of one to three hydroxyl groups, a pyridyl group optionally having substituents, and thienyl group optionally having substituents; and
G represents a hexose group which may be substituted with an amino group.

40. The method of claim 38, wherein X¹ represents 2-hydroxyl group, X² represents 10-hydroxyl group, R represents (1-hydroxymethyl-2-hydroxyl) ethylamino group, and G represents β-D-glucopyranosyl.


SEQUENCE LISTING (17 sequences)

SEQ ID NO: 1 - DNA, 2027 bp, Homo sapiens
  CDS (45)..(2009)
ttaagaccgagctctattaagctgaaaagataaaaactctccagatgtcttccagtaatgtcgaagtttttatcccagtgtcacaaggaaacaccaatggcttccccgcgacagcttccaatgacctgaaggcatttactgaaggagctgtgttaagttttcataacatctgctatcgagtaaaactgaagagtggctttctaccttgtcgaaaaccagttgagaaagaaatattatcgaatatcaatgggatcatgaaacctggtctcaacgccatcctgggacccacaggtggaggcaaatcttcgttattagatgtcttagctgcaaggaaagatccaagtggattatctggagatgttctgataaatggagcaccgcgacctgccaatttcaaatgtaattcaggttacgtggtacaagatgatgttgtgatgggcactctgacggtgagagaaaacttacagttctcagcagctcttcggcttgcaacaactatgacgaatcatgaaaaaaacgaacggattaacagggtcattcaagagttaggtctggataaagtggcagactccaaggttggaactcagtttatccgtggtgtgtctggaggagaaagaaaaaggactagtataggaatggagcttatcactgatccttccatcttgttcttggatgagcctacaactggcttagactcaagcacagcaaatgctgtccttttgctcctgaaaaggatgtctaagcagggacgaacaatcatcttctccattcatcagcctcgatattccatcttcaagttgtttgatagcctcaccttattggcctcaggaagacttatgttccacgggcctgctcaggaggccttgggatactttgaatcagctggttatcactgtgaggcctataataaccctgcagacttcttcttggacatcattaatggagattccactgctgtggcattaaacagagaagaagactttaaagccacagagatcatagagccttccaagcaggataagccactcatagaaaaattagcggagatttatgtcaactcctccttctacaaagagacaaaagctgaattacatcaactttccgggggtgagaagaagaagaagatcacagtcttcaaggagatcagctacaccacctccttctgtcatcaactcagatgggtttccaagcgttcattcaaaaacttgctgggtaatccccaggcctctatagctcagatcattgtcacagtcgtactgggactggttataggtgccatttactttgggctaaaaaatgattctactggaatccagaacagagctggggttctcttcttcctgacgaccaaccagtgtttcagcagtgtttcagccgtggaactctttgtggtagagaagaagctcttcatacatgaatacatcagcggatactacagagtgtcatcttatttccttggaaaactgttatctgatttattacccatgaggatgttaccaagtattatatttacctgtatagtgtacttcatgttaggattgaagccaaaggcagatgccttcttcgttatgatgtttacccttatgatggtggcttattcagccagttccatggcactggccatagcagcaggtcagagtgtggtttctgtagcaacacttctcatgaccatctgttttgtgtttatgatgattttttcaggtctgttggtcaatctcacaaccattgcatcttggctgtcatggcttcagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaaatttccccttaattc

SEQ ID NO: 2 - protein, 655 aa, Homo sapiens
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 3 - DNA, 63 bp, Artificial Sequence
  artificially synthesized primer
ggccagtgaattgtaatacgactcactatagggaggcggtttttttttttttttttttttttt

SEQ ID NO: 4 - DNA, 26 bp, Artificial Sequence
  artificially synthesized primer
ctcatttaaaaacttgctcgggaacc

SEQ ID NO: 5 - DNA, 26 bp, Artificial Sequence
  artificially synthesized primer
caagaggccagaaaagagcatcataa

SEQ ID NO: 6 - DNA, 21 bp, Artificial Sequence
  artificially synthesized primer
tactggggcttattattggtg

SEQ ID NO: 7 - DNA, 24 bp, Artificial Sequence
  artificially synthesized primer
aaaagcgattgtcatgagaagtgt

SEQ ID NO: 8 - DNA, 30 bp, Artificial Sequence
  artificially synthesized primer
caaaaagcttaagaccgagctctattaagc

SEQ ID NO: 9 - DNA, 31 bp, Artificial Sequence
  artificially synthesized primer
atcctctagaccaggtttcatgatcccattg

SEQ ID NO: 10 - DNA, 23 bp, Artificial Sequence
  artificially synthesized primer
gaattaaggggaaatttaagaat

SEQ ID NO: 11 - DNA, 24 bp, Artificial Sequence
  artificially synthesized primer
agagatcgatgccctgctttacca

SEQ ID NO: 12 - DNA, 2053 bp, Artificial Sequence
  ABCG2 482Tmutant sequence
ctattaagctgaaaagataaaaactctccagatgtcttccagtaatgtcgaagtttttatcccagtgtcacaaggaaacaccaatggcttccccgcgacagcttccaatgacctgaaggcatttactgaaggagctgtgttaagttttcataacatctgctatcgagtaaaactgaagagtggctttctaccttgtcgaaaaccagttgagaaagaaatattatcgaatatcaatgggatcatgaaacctggtctcaacgccatcctgggacccacaggtggaggcaaatcttcgttattagatgtcttagctgcaaggaaagatccaagtggattatctggagatgttctgataaatggagcaccgcgacctgccaatttcaaatgtaattcaggttacgtggtacaagatgatgttgtgatgggcactctgacggtgagagaaaacttacagttctcagcagctcttcggcttgcaacaactatgacgaatcatgaaaaaaacgaacggattaacagggtcattcaagagttaggtctggataaagtggcagactccaaggttggaactcagtttatccgtggtgtgtctggaggagaaagaaaaaggactagtataggaatggagcttatcactgatccttccatcttgttcttggatgagcctacaactggcttagactcaagcacagcaaatgctgtccttttgctcctgaaaaggatgtctaagcagggacgaacaatcatcttctccattcatcagcctcgatattccatcttcaagttgtttgatagcctcaccttattggcctcaggaagacttatgttccacgggcctgctcaggaggccttgggatactttgaatcagctggttatcactgtgaggcctataataaccctgcagacttcttcttggacatcattaatggagattccactgctgtggcattaaacagagaagaagactttaaagccacagagatcatagagccttccaagcaggataagccactcatagaaaaattagcggagatttatgtcaactcctccttctacaaagagacaaaagctgaattacatcaactttccgggggtgagaagaagaagaagatcacagtcttcaaggagatcagctacaccacctccttctgtcatcaactcagatgggtttccaagcgttcattcaaaaacttgctgggtaatccccaggcctctatagctcagatcattgtcacagtcgtactgggactggttataggtgccatttactttgggctaaaaaatgattctactggaatccagaacagagctggggttctcttcttcctgacgaccaaccagtgtttcagcagtgtttcagccgtggaactctttgtggtagagaagaagctcttcatacatgaatacatcagcggatactacagagtgtcatcttatttccttggaaaactgttatctgatttattacccatgacgatgttaccaagtattatatttacctgtatagtgtacttcatgttaggattgaagccaaaggcagatgccttcttcgttatgatgtttacccttatgatggtggcttattcagccagttccatggcactggccatagcagcaggtcagagtgtggtttctgtagcaacacttctcatgaccatctgttttgtgtttatgatgattttttcaggtctgttggtcaatctcacaaccattgcatcttggctgtcatggcttcagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaaatttccccttaattcaagggcaattctgcagatatccagcacagtggcggccgc

SEQ ID NO: 13 - protein, 655 aa, Artificial Sequence [sequence omitted]

SEQ ID NO: 14 - DNA, 23 bp, Artificial Sequence
  artificially synthesized primer
ccatgacgatgttaccaagtatt

SEQ ID NO: 15 - DNA, 23 bp, Artificial Sequence
  artificially synthesized primer
aacatcgtcatgggtaataaatc

SEQ ID NO: 16 - DNA, 23 bp, Artificial Sequence
  artificially synthesized primer
cattcatcagcctcgatattcca

SEQ ID NO: 17 - DNA, 22 bp, Artificial Sequence
  artificially synthesized primer
accacactctgacctgctgcta